SEQUENCE LISTING (16 sequences)

SEQ ID NO: 1 - DNA, 20 bp, Artificial sequence
  Synthetic primer external-1 Pax7
gaaccacatccgtcacaaga

SEQ ID NO: 2 - DNA, 20 bp, Artificial sequence
  Synthetic primer internal-1 Pax7
tttcccatggttgtgtctcc

SEQ ID NO: 3 - DNA, 20 bp, Artificial sequence
  Synthetic primer external-2 Pax7
gagcactcggctaatcgaac

SEQ ID NO: 4 - DNA, 18 bp, Artificial sequence
  Synthetic primer internal-2 Pax7
gtcgcagtgaccgtcctt

SEQ ID NO: 5 - DNA, 20 bp, Artificial sequence
  Synthetic primer external-1 Pax3
aaccatatccgccacaagat

SEQ ID NO: 6 - DNA, 20 bp, Artificial sequence
  Synthetic primer internal-1 Pax3
aaacccaagcaggtgacaac

SEQ ID NO: 7 - DNA, 20 bp, Artificial sequence
  Synthetic primer external-2 Pax3
ctagatccgcctcctcctct

SEQ ID NO: 8 - DNA, 20 bp, Artificial sequence
  Synthetic primer internal-2 Pax3
ggatgcggctgatagaactc

SEQ ID NO: 9 - DNA, 20 bp, Artificial sequence
  Synthetic primer external-1 Myf5
agacgcctgaagaaggtcaa

SEQ ID NO: 10 - DNA, 20 bp, Artificial sequence
  Synthetic primer internal-1 Myf5
ccaccaaccctaaccagaga

SEQ ID NO: 11 - DNA, 20 bp, Artificial sequence
  Synthetic primer external-2 Myf5
agctggacacggagctttta

SEQ ID NO: 12 - DNA, 20 bp, Artificial sequence
  Synthetic primer internal-2 Myf5
ctgttctttcgggaccagac

SEQ ID NO: 13 - DNA, 20 bp, Artificial sequence
  Synthetic primer external-1 MyoD
agcgcaagaccaccaacgct

SEQ ID NO: 14 - DNA, 19 bp, Artificial sequence
  Synthetic primer internal-1 MyoD
gccttctacgcacctggac

SEQ ID NO: 15 - DNA, 20 bp, Artificial sequence
  Synthetic primer external-2 MyoD
gtggagatgcgctccactat

SEQ ID NO: 16 - DNA, 20 bp, Artificial sequence
  Synthetic primer internal-2 MyoD
actcttccctggcctggact

(19) United States
(12) Patent Application Publication
(10) Pub. No.: US 2010/0055039 A1
(43) Pub. Date: Mar. 4, 2010

(54) NON-INVASIVE METHODS OF MONITORING ENGRAFTED STEM CELLS AND METHODS FOR ISOLATION OF SKELETAL MUSCLE STEM CELLS

(76) Inventors: Regis Doyonnas, Stonington, CT (US); Alessandra Sacco, Mountain View, CA (US); Helen M. Blau, Menlo Park, CA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/554,118

(22) Filed: Sep. 4, 2009

Related U.S. Application Data
(60) Provisional application No. 61/094,254, filed on Sep. 4, 2008.
(60) Provisional application No. 61/192,116, filed on Sep. 15, 2008.

(51) Int. Cl. A61K 49/00

(52) U.S. Cl. 424/9.6; 424/9.1; 435/378; 435/325

(57) ABSTRACT

The embodiments of the present disclosure encompass methods for non-invasive in vivo bioluminescence imaging that allow the dynamics of stem cell behavior to be followed in a manner not possible using conventional retrospective static histological analyses. By imaging luciferase-generated bioluminescence activity emanating from isolated stem cells, for example, real time quantitative and kinetic analyses can show that donor-derived muscle stem cells may proliferate and engraft rapidly after injection until homeostasis is reached. In addition, the response of the stem cells to injury and participation in the regenerative response can be monitored over time. Other aspects of the disclosure encompasses methods for determining the suitability of a stem cell for tissue replacement, methods for repairing muscle injury, and methods for isolating muscle stem cells from a tissue sample.

### CROSS-REFERENCE TO RELATED APPLICATIONS

This application claims priority to U.S. Provisional Patent Application Ser. No. 61/094,254, entitled “NON-INVASIVE METHODS OF MONITORING ENGRAFTED SATELLITE CELLS” filed on Sep. 4, 2008, and U.S. Provisional Patent Application Ser. No. 61/112,116, entitled “ISOLATION OF SKELETAL MUSCLE STEM CELLS AND NON-INVASIVE METHODS OF MONITORING ENGRAFTED STEM CELLS DELIVERED TO TISSUES” filed on Sep. 15, 2008, the entireties of which are hereby incorporated by reference.

### TECHNICAL FIELD

The present disclosure is generally related to methods of isolating a subset of skeletal muscle satellite cells from the muscle tissues of a mammal. The present disclosure is further generally related to non-invasive methods of time-extended monitoring of stem cells engrafted into the solid tissues of a subject mammal.

### SEQUENCE LISTING

The present disclosure includes a sequence listing incorporated herein by reference in its entirety.

### BACKGROUND

Adult muscle satellite cells play a major role in postnatal skeletal muscle growth and regeneration (Charge & Rudnicki, Physiol. Rev. 84: 209 (2004). Satellite cells reside as quiescent cells underneath the basal lamina that surrounds muscle fibers (Mauro, J. Biophys. Biochem. Cytol. 9: 493 (1961)) and respond to damage by giving rise to transient amplifying cells (progenitors) and myoblasts that fuse with myofibers. Recent ground-breaking experiments showed that in contrast to cultured myoblasts, freshly isolated FACS-sorted satellite cells (Montarras et al., Science 309: 2064 (2005); Kuang et al., Cell 129: 999 (2007), or satellite cells derived from the transplantation of one intact myofiber (Collins et al., Cell 122: 289 (2005)) contribute robustly to muscle repair. However, since satellite cells are known to comprise a heterogeneous population (Kuang et al., Cell 129: 999 (2007); Sherwood et al., Cell 119: 543 (2004)), a clonal analysis is required to demonstrate stem cell function and to identify the stem cell within the satellite cell population.

The ability to detect engrafted muscle stem cells, as well as stem cells isolated from neural, pancreatic tissues and the like, and to monitor their function in vivo is currently restricted to static histological images that provide a snapshot of the degree of participation of the cells in a given tissue at a given time. Using such classical histological methods, the contribution of the stem cells to adult tissues is difficult to quantify, preventing efficiency comparisons between different putative stem cell types, methods of stem cell delivery, and their function in animal models of disease or injury. In addition, analyses of stem cell contributions to solid tissues are cumbersome and expensive, requiring numerous mice, as for each time point the sacrifice of several animals is necessary.

### SUMMARY

The embodiments of the present disclosure encompass methods for non-invasive in vivo bioluminescence imaging that allow the dynamics of stem cell behavior to be followed in a manner not possible using conventional retrospective static histological analyses. By imaging luciferase-generated bioluminescence activity emanating from isolated stem cells, for example, real time quantitative and kinetic analyses can show that donor-derived muscle stem cells may proliferate and engraft rapidly after injection until homeostasis is reached. In addition, the response of the stem cells to injury and participation in the regenerative response can be monitored over time.

One aspect of the present disclosure, therefore, encompasses non-invasive methods for determining the proliferative status of engrafted stem cells in a recipient subject mammal, comprising: providing an isolated stem cell or a population of stem cells, wherein the stem cell or population of stem cells expresses a heterologous reporter; delivering the isolated stem cell or population of stem cells to a subject mammal; and non-invasively detecting the reporter in the recipient subject mammal, thereby detecting the population of engrafted stem cells, or progeny thereof, in the subject mammal.

In embodiments of this aspect of the disclosure, the isolated stem cell or population of stem cells may be obtained from a transgenic animal that comprises a heterologous nucleic acid encoding the reporter operably linked to a promoter driving expression of the heterologous nucleic acid.

In embodiments of this aspect of the disclosure, the step of providing an isolated stem cell or a population of stem cells can further comprise the step of transfecting a stem cell or population of stem cells with a heterologous nucleic acid encoding the reporter, wherein the reporter is operably linked to a promoter driving expression of the heterologous nucleic acid, and wherein the isolated stem cell or population of stem cells is transfected with the heterologous nucleic acid after isolation from a mammal.

In embodiments of the disclosure, the isolated stem cell, or population of stem cells can be selected from the group consisting of: a mesenchymal stem cell, a hematopoietic stem cells, a neural crest stem cell, a placental stem cell, an embryonic stem cell, and a mesodermal stem cell. In some embodiments, the isolated stem cell, or population of stem cells, is a subset of muscle satellite cell(s) isolated from a muscle tissue.

In embodiments of the disclosure, the reporter encoded by the heterologous nucleic acid can be a bioluminescent reporter, a fluorescent reporter, a PET reporter, or a combination thereof. In some embodiments of the disclosure, the bioluminescent reporter is a luciferase.

In other embodiments of this aspect of the disclosure, the isolated stem cell can be a single stem cell isolated from a population of isolated cells by delivery into a microwell imprinted in a hydrogel.

In embodiments of this aspect of the disclosure where the reporter is a luciferase, the method can further comprise: administering to the subject mammal a bioluminescence initiator, whereupon interaction of the bioluminescence initiator with the luciferase causes the luciferase to emit bioluminescence; and detecting the emitted bioluminescence, thereby detecting the presence of a population of stem cells in the subject.

In embodiments of the methods of this aspect of the disclosure, the method may further comprise measuring the intensity of the bioluminescence, where the intensity of the bioluminescence indicates the number of stem cells in the subject mammal. In these embodiments, the method can further comprise: measuring a first bioluminescence intensity; delivering to the subject mammal a test compound; and measuring a second bioluminescence intensity, where a difference in the first and the second bioluminescence intensities can indicate that the test compound modulates the proliferation of the stem cell or stem cell population delivered to the subject mammal.

Another aspect of the disclosure encompasses methods for determining the suitability of a stem cell for tissue replacement, comprising: obtaining a population of isolated candidate stem cells; genetically modifying a proportion of the population of candidate stem cells with a heterologous nucleic acid encoding a reporter polypeptide, where the heterologous nucleic acid can be under the expression control of a promoter selected from the group consisting of: a constitutive promoter, an inducible promoter, a stem cell-specific promoter, and a tissue specific promoter, and wherein the heterologous nucleic acid is integrated into the genome of the cells; engrafting the genetically modified candidate stem cells to a subject mammal tissue; inducing the emission of a detectable signal by the engrafted cells in the subject mammal; and determining from the intensity of the detectable signal the degree of proliferation of said cells in the subject mammal tissue, thereby indicating the suitability of the isolated cells for tissue replacement.

Yet another aspect of the disclosure encompasses methods method for repairing muscle injury, comprising: obtaining a population of muscle satellite cells; isolating from the population of muscle satellite cells a subset population having stem cell activity and regenerative capacity by: genetically modifying a proportion of the muscle satellite cells with a heterologous nucleic acid encoding a reporter polypeptide, where the heterologous nucleic acid is under the expression control of a promoter selected from the group consisting of: a constitutive promoter, an inducible promoter, a stem cell-specific promoter, and a tissue specific promoter, and where the heterologous nucleic acid is integrated into the genome of the cells; engrafting the genetically modified muscle satellite cells to a subject mammal tissue; inducing the emission of a detectable signal by the engrafted cells in the subject mammal; determining from the intensity of the detectable signal, the degree of proliferation of said cells in the subject mammal tissue, thereby indicating the suitability of the isolated muscle satellite cells for tissue replacement; and delivering to a site of muscle injury in a subject mammal the isolated subset population of muscle satellite cells having muscle stem cell characteristics, whereupon the subset population proliferates and differentiates into myoblasts and muscle fibers to an amount that repairs the site of the injury.

Still yet another aspect of the present disclosure encompasses methods for isolating muscle stem cells from a tissue sample, comprising: obtaining from a subject animal or human a muscle tissue sample; obtaining a population of cells in suspension from the tissue sample; contacting the population of cells in suspension with a first panel of antibody species, where each species of the first panel of antibody species selectively binds to a cell surface antigen not located on a muscle stem cell surface; partitioning the muscle cells binding to the first panel of antibodies from the population of cells in suspension; contacting the population of muscle cells in suspension with a second panel of antibody species, where each species of the second panel of antibody species selectively binds to a muscle stem cell-specific surface antigen; isolating muscle stem cells from the population of cells in suspension by partitioning cells binding to the second panel of antibodies, where the partitioned cells are muscle stem cells.

In embodiments of this aspect of the disclosure, the first panel of antibody species can comprise at least one antibody species selected from the group consisting of: an anti-CD45 antibody, an anti-CD11b antibody, an anti-CD31 antibody, and an anti-Sca1 antibody.

In embodiments of this aspect of the disclosure, the second panel of antibodies comprises an anti-α7 integrin antibody, an anti-CD34 antibody, or an anti-α7 integrin antibody, and an anti-CD34 antibody.

In embodiments of this aspect of the disclosure, the isolated muscle stem cells can be CD45⁻, CD11b⁻, CD31⁻, Sca1⁻, α7 integrin⁺, and CD34⁺.

In embodiments of this aspect of the disclosure, the tissue sample can obtained from a transgenic animal, where the cells of the transgenic animal comprise a heterologous nucleic acid encoding a reporter polypeptide operably linked to a promoter driving expression of the heterologous nucleic acid. In some embodiments of this aspect of the disclosure, the method may further comprise isolating a single muscle stem cell from a population of isolated cells by delivery into a microwell imprinted in a hydrogel.

Yet another aspect of the disclosure encompasses an isolated muscle stem cell, or a population of isolated muscle stem cells, where the isolated muscle stem cell, or population of muscle stem cells are characterized as CD45⁻, CD11b⁻, CD31⁻, Sca1⁻, α7 integrin⁺, and CD34⁺, and where the isolated muscle stem cell, or population of muscle stem cells when implanted into a recipient animal proliferate therein to form a population of engrafted stem cells.

In embodiments of this aspect of the disclosure, the isolated muscle stem cell, or a population of isolated muscle stem cells, when implanted into a recipient subject mammal, differentiates into muscle cells.

### BRIEF DESCRIPTION OF THE DRAWINGS

The drawings are described in greater detail in the description and examples below.

Further aspects of the present disclosure will be more readily appreciated upon review of the detailed description of its various embodiments, described below, when taken in conjunction with the accompanying drawings.

FIGS. 1A-1G illustrate the isolation and characterization of the α7integrin⁺CD34⁺ cell fraction as muscle stem cells (muscle stem cells).

FIG. 1A illustrates a flow cytometry analysis of freshly isolated muscle cells. Within a first sorting phase, living cells were gated for forward scatter (FSC) and PI negativity (left panel), then cells negative for blood markers CD45 and CD11b, for endothelial marker CD31 and for mesenchymal marker Sca1 were gated (middle panel), and within this population the α7integrin⁺CD34⁺ fraction was sorted (right panel). In a second sorting phase, the α7integrin⁺CD34⁺ fraction was resorted.

FIGS. 1B and 1C are digital images illustrating the results when, 48 hrs after isolation, muscle α7integrin⁺CD34⁺ cells were stained for Pax7 (FIG. 1B: Pax7; FIG. 1C, ToPro, nuclei). Scale bars=80 μm.

FIG. 1D is a digital image illustrating that after 5 days of culture in growth medium, α7integrin⁺CD34⁺ cells isolated from Myf5-nLacZ transgenic mice exhibited β-galactosidase activity. Scale bars=80 μm.

FIG. 1E is a digital image illustrating that after 3 days in culture in differentiation medium, α7integrin⁺CD34⁺ (from GFP transgenic mice) differentiated to form myotubes that expressed myogenin (lightest areas). Scale bars=80 μm.

FIGS. 1F and 1G are digital images illustrating freshly isolated α7integrin⁺CD34⁺ cells from Myf5-nLacZ mice transplanted into recipient animals. One month after transplant, recipient muscles were damaged by notexin (NTX) injection and 5 days later, immunofluorescence of transverse tissue sections revealed cells engrafted in the satellite cell position (arrowhead and insert). Scale bars=20 μm.

FIGS. 2A-2F illustrate satellite cell engraftment monitored by in vivo non-invasive bioluminescence imaging.

FIG. 2A (upper) is a graph illustrating the relationship between the increasing number of myoblasts injected into the Tibialis Anterior muscles (TAs) of NOD/SCID recipients, and the level of bioluminescence when imaged 2 hours after injection. Bioluminescence data is represented as average±s.e.m. (n=4; P<0.05). FIG. 2A (lower) shows digital bioluminescent images of representative injected mice. The number of cells injected is indicated above the images. (Gray scale on the right: minimum, 10⁴ photons cm⁻² sec⁻¹, maximum, 10⁵ photons cm⁻² sec⁻¹).

FIG. 2B is a digital image of when freshly isolated satellite cells (5,000 cells) or cultured primary myoblasts (20,000 cells) from double transgenic mice were injected into recipients and imaged 4 weeks after transplantation (n=4; color scale on the right, minimum, 0.5×10⁵ photons cm⁻² sec⁻¹, maximum, 15.0×10⁵ photons cm⁻² sec⁻¹).

FIG. 2C is a digital photomicrograph showing luciferase-generated immunofluorescence of expression in transverse muscle sections from the mice shown in FIG. 2B and revealing the contribution of muscle satellite cells, and not of myoblasts, to muscle fibers. Scale bars=100 μm.

FIG. 2D is a digital photomicrograph showing β-galactosidase staining of Myf5⁺ cells in muscles transplanted with satellite cells (5 days before tissue harvesting, muscles were damaged with NTX). No Myf5⁺ cells were detected in muscles transplanted with myoblasts (right image). Scale bars=100 μm.

FIG. 2E shows a pair of graphs showing the results from when different numbers of satellite cells were injected into muscles of recipient animals, and engraftment was measured by imaging recipient animals 4 weeks after transplantation (non-injected legs are shown as negative control, Ctrl). A scattered graph of bioluminescent values of individual mice is shown (top), and a histogram graph (bottom) showing percentages of mice exhibiting successful engraftment for each number of cells injected.

FIG. 2F is a graph showing engraftment of satellite cells (5,000, 500, and 10) and monitoring by imaging over a period of 6 weeks after transplantation (average±s.e.m.)(n=3; P<0.05).

FIGS. 3A-3C illustrates satellite cell proliferation following muscle tissue damage.

FIG. 3A is a graph showing a low number of satellite cells (10-500) transplanted into recipients on day 0. After 49 days, the TA muscles of one group were damaged by NTX injection, resulting in a substantial increase in cell numbers. At 96 days, a second NTX injection led to a second increase in cell numbers, while in the undamaged group no significant change was detected. Average bioluminescence fold increase±s.e.m. is shown (n=5; P<0.05).

FIG. 3B shows a series of digital bioluminescent images showing a representative NTX-damaged animal acquired on the days indicated (top) (gray scale on the right: minimum, 10⁴ photons cm⁻² sec⁻¹, maximum, 3×10⁵ photons cm⁻² sec⁻¹)

FIG. 3C is a graph illustrating that when high numbers of primary myoblasts (4×10⁵) are transplanted into recipients followed by bioluminescence imaging (results from mice transplanted with 10-500 satellite cells are shown for comparison). At 3 weeks post transplant, recipient muscles were damaged by NTX injection. Average bioluminescence fold increase±s.e.m. is shown (n=6).

FIGS. 4A-4D illustrate that the transplantation of single satellite cells demonstrates self-renewal function.

FIG. 4A schematically illustrates single satellite cell transplantation: cells were isolated from Myf5-nLacZ/Fluc double transgenic mice by FACS, segregated as single cells in hydrogel microwells (Scale bars=150 μm) and individually picked by micromanipulation 2 hours later.

FIGS. 4B is a graph illustrating that 3 of 72 single cell transplants resulted in engraftment above background, detected by imaging mice 4 weeks after transplantation. The digital bioluminescent images of the 3 positive recipients after single satellite cell transplantation (gray scale on the right: minimum, 0.8×10⁴ photons cm⁻² sec⁻¹; maximum, 30.0×10⁴ photons cm⁻² sec⁻¹).

FIG. 4C shows the results of a similar experiment to that shown in FIG. 4B.

FIG. 4D are digital photomicrographs illustrating the detection of muscle cells re-isolated from mice transplanted with single cells and donor-derived luciferase⁺Pax7⁺ cells. Scale bars=100 μm.

FIG. 4E shows a digital image of the immunofluorescence of luciferase expression in transverse muscle sections from mice shown in FIGS. 4B and 4C, showing the contribution of single muscle stem cell progeny to muscle fibers.

FIGS. 5A and 5B illustrate linearity of bioluminescence imaging in vitro.

FIG. 5A shows a series of digital images illustrating primary myoblasts isolated from Myf5-nLacZ/Fluc double transgenic mice (β-galactosidase, left panel, and luciferase, right panel). Scale bars=100 μm.

FIG. 5B (left) is a digital bioluminescence image showing increasing numbers of Myf5-nLacZ/Fluc myoblasts plated in a 96-well plate. Imaging was performed immediately after plating. The image is of the 96-well plate. The number of cells plated is indicated above the image. (Gray scale: minimum, 1.0×10⁵ photons cm⁻² sec⁻¹, maximum, 18.0×10⁵ photons cm⁻² sec⁻¹).

FIG. 5B (right) is a graph showing bioluminescence data represented as average±s.e.m. (n=5; P<0.0001).

FIG. 6A is a graph illustrating the proliferation of muscle stem cells in response to serial tissue damage as indicated by a bioluminescence fold increase above engraftment level.

FIG. 6B shows a series of graphs showing the absolute bioluminescence measurements for individual mice (5 controls and 5 NTX-damaged) assayed 17 times over a 70 day time course. As engraftment levels differed among different mice, a y-axis scale for each bar graph was selected to best represent the data.

FIG. 7A shows digital images illustrating the time-course of muscle regeneration by endogenous and transplanted cells following irradiation and NTX damage of TA muscles of NOD/SCID mice. Legs of NOD/SCID mice were irradiated with 18Gy. Two months later, Tibialis Anterior muscles were damaged with NTX and tissue harvested at the indicated days. In these experimental conditions, regeneration was still ongoing 13 and 19 days post damage in irradiated tissues. Scale bars=100 μm.

FIG. 7B (top panels) shows digital images illustrating the time-course of muscle regeneration by transplanted cells following irradiation and NTX damage of TA muscles of NOD/SCID mice. Legs of NOD/SCID mice were irradiated with 18Gy and 500 muscle stem cells from Myf5nLacZ/FLuc double transgenic mice were transplanted. After engraftment (7 weeks), muscles were damaged with NTX and harvested at the days indicated. β-galactosidase histochemistry (shown in the top panels) showed donor-derived Myf5-β-gal⁺ cells at days 7 and 13, indicative of ongoing regeneration. Scale bars=120 μm. FIG. 7B (lower) shows a graph showing number of β-galactosidase⁺ cells after NTX damage at days 7, 13 and 19 (average±s.e.m.) (n=4, *P<0.05), showing that some Myf5-βgal⁺ cells could still be detected at days 13 and 19 post injury, indicating ongoing regeneration.

FIG. 7C shows digital images illustrating the time-course of muscle regeneration by endogenous and transplanted cells following irradiation and NTX damage of TA muscles of NOD/SCID mice, and showing that in this experimental setting muscle regeneration takes place during a period of 2-3 weeks. Legs of NOD/SCID mice were irradiated with 18Gy and muscle satellite cells (muscle stem cells) derived from GFP/FLuc double transgenic mice were transplanted. After engraftment (7 weeks), muscles were damaged with NTX and harvested at the indicated days. Immunofluorescence for embryonic myosin heavy chain (eMyHC) (a myosin isoform that is transiently expressed during adult skeletal muscle regeneration) shows that regeneration was continuing at days 13 and 19, as eMyHC⁺ myofibers could be detected at these time points. Scale bars=50 μm.

FIG. 8A is a panel of digital photomicrographs showing cell proliferation during muscle regeneration.

FIG. 8B is a graph showing quantification of donor-derived (GFP⁺) proliferating (Ki67⁺) cells at day 7, 13 and 19 after NTX damage (average±s.e.m) (n=3, *P<0.05).

FIG. 9A is a panel of digital photomicrographs showing apoptosis increases over time during NTX-induced muscle regeneration

FIG. 9B is a graph showing quantification of apoptotic cells (TUNEL⁺) during muscle regeneration.

FIG. 10 is a graph showing that luciferase activity is not significantly different between proliferating myoblasts and mature muscle fibers. Bioluminescence values/μg DNA represented as average±s.e.m. (n=4; P>0.05).

FIG. 11 schematically represents the dynamics of muscle stem cell behavior in vivo during three waves of proliferation, which correlates with the time course of classical histological methods (Ki67, TUNEL, embryonic myosin heavy chain, myf5 lacz), and with the bioluminescence imaging shown in FIGS. 2E, 3A, and 3B.

FIG. 12 illustrates that single muscle stem cells (1-40) were individually sorted and reverse transcribed followed by polymerase chain reaction. The results show that this population consistently and homogeneously expresses Pax7 and Myf5, the expected transcriptional profile for satellite cells. In contrast, both MyoD and Pax3 expression are heterogeneous, indicating the presence of committed progenitors.

FIG. 13A illustrates the results of transplanting freshly isolated muscle stem cells or cultured myoblasts into recipients and engraftment was monitored by imaging over a period of six weeks after transplantation. FIG. 13A (left panel) shows a digital bioluminescent images of representative injected mice acquired at the indicated days are shown (bioluminescence values are indicated as photons cm⁻² sec⁻¹×10⁴). FIG. 13A (right) shows a graph of bioluminescence measurements (average±s.e.m., n=5; P<0.05).

FIG. 13B illustrates muscle stem cells from GFP/FLuc transgenic mice transplanted into recipients. Four weeks later, mice were analyzed by bioluminescence imaging and for histology. Regression analysis (FIG. 13B, left) shows a significant (n=10, P<0.0001) correlation between the number of GFP1 myofibers and luciferase activity in individual mice. Representative digital images of immunofluorescence and bioluminescence imaging (FIG. 13B, right). Bioluminescence values are indicated as photons cm⁻² sec⁻¹×10⁵). Scale bar=120 μm

The details of some exemplary embodiments of the methods and systems of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. Patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

In describing and claiming the disclosed subject matter, the following terminology will be used in accordance with the abbreviations and definitions as set forth below.

### Abbreviations

GFP, green fluorescent protein; NTX, notexin; SCID, NOD/SCID, non-obese diabetic/severe combined immunodeficiency disease; TA, tibalis anterior (muscle).

### Definitions

The term “subject mammal” is used herein to include all mammals, including humans. It also includes an individual animal in all stages of development, including embryonic and fetal stages.

The term “stem cell” as used herein refers to a mammalian cell that has the ability both to self-renew, and to generate differentiated progeny (see, for example, Morrison et al. (1997) Cell 88:287-298). Stem cells are primal cells found in all multi-cellular organisms. They retain the ability to renew themselves through mitotic cell division and can differentiate into a diverse range of specialized cell types. Stem cells include but are not limited to, mesenchymal stem cells, hematopoietic stem cells, neural crest stem cells, placental stem cells, embryonic stem cells, and mesodermal stem cells, among others. Mesenchymal stem cells (MSC) are pluripotent blast cells found inter alia in bone marrow, blood dermis and periosteum and are capable of differentiating into any of the specific types of mesenchymal stem or connective tissue cells, including adipose, osseous (including osteoblasts) cartilaginous, elastic, and fibrous connective tissues.

The three broad categories of mammalian stem cells are: embryonic stem cells, derived from blastocysts, adult stem cells, which are found in adult tissues, and cord blood stem cells, which are found in the umbilical cord. In a developing embryo, stem cells can differentiate into all of the specialized embryonic tissues. In adult organisms, stem cells and progenitor cells act as a repair system for the body, replenishing specialized cells.

As stem cells can be grown and transformed into specialized cells with characteristics consistent with cells of various tissues such as muscles or nerves through cell culture, their use in medical therapies has been proposed. In particular, embryonic cell lines, autologous embryonic stem cells generated through therapeutic cloning, and highly plastic adult stem cells from the umbilical cord blood or bone marrow are touted as promising candidates. The term “undifferentiated” as used herein refers to pluripotent embryonic stem cells which have not developed a characteristic of a more specialized cell. As will be recognized by one of skill in the art, the terms “undifferentiated” and “differentiated” are relative with respect to each other. A stem cell which is “differentiated” has a characteristic of a more specialized cell, such as but not limited to a muscle cell. Differentiated and undifferentiated cells are distinguished from each other by several well-established criteria, including morphological characteristics such as relative size and shape, ratio of nuclear volume to cytoplasmic volume; and expression characteristics such as detectable presence of known markers of differentiation. A marker of differentiation indicating that cells are differentiated or undifferentiated includes a protein, carbohydrate, lipid, nucleic acid, functional characteristic and/or morphological characteristic which is specific to a differentiated cell.

The term “muscle cell” as used herein refers to any cell which contributes to muscle tissue. Myoblasts, satellite cells, myotubes, and myofibril tissues are all included in the term “muscle cells”. Muscle cell effects may be induced within skeletal, cardiac and smooth muscles. Muscle tissue in adult vertebrates will regenerate from reserve myoblasts called “satellite cells”. Satellite cells are distributed throughout muscle tissue and are mitotically quiescent in the absence of injury or disease. Following muscle injury or during recovery from disease, satellite cells will reenter the cell cycle, proliferate and 1) enter existing muscle fibers or 2) undergo differentiation into multinucleate myotubes which form new muscle fiber. The myoblasts ultimately yield replacement muscle fibers or fuse into existing muscle fibers, thereby increasing fiber girth by the synthesis of contractile apparatus components. This process is illustrated, for example, by the nearly complete regeneration which occurs in mammals following induced muscle fiber degeneration; the muscle progenitor cells proliferate and fuse together regenerating muscle fibers.

The terms “suspension of cells” or “cells in suspension” as used herein refer to the cells that do not adhere to a solid substratum.

The terms “primary culture” and “primary cells” refer to cells derived from intact or dissociated tissues or organ fragments. A culture is considered primary until it is passaged (or subcultured) after which it is termed a “cell line” or a “cell strain.” The term “cell line” does not imply homogeneity or the degree to which a culture has been characterized. A cell line is termed “clonal cell line” or “clone” if it is derived from a single cell in a population of cultured cells. Primary cells can be obtained directly from a human or animal adult or fetal tissue, including blood. The primary cells may comprise a primary cell line, or such as, but not limited to, a population of muscle satellite cells.

The terms “grafting”, “engrafting”, and “transplanting” and “graft” and “transplantation” as used herein refer to the process by which embryonic-like stem cells or other cells according to the present disclosure are delivered to the site where the cells are intended to exhibit a favorable effect, such as repairing damage to a patient's central nervous system, treating autoimmune diseases, treating diabetes, treating neurodegenerative diseases, or treating the effects of nerve, muscle and/or other damage caused by birth defects, stroke, cardiovascular disease, a heart attack or physical injury or trauma or genetic damage or environmental insult to the body, caused by, for example, disease, an accident or other activity. The stem cells or other cells for use in the methods of the present disclosure can also be delivered in a remote area of the body by any mode of administration as described above, relying on cellular migration to the appropriate area in the body to effect transplantation. For example, the term “cell engraftment” as used herein can refer to the process by which cells such as, but not limited to, muscle stem cells, are delivered to, and become incorporated into, a differentiated tissue such as a muscle, and become part of that tissue. For example, muscle stem cells, when delivered to a muscle tissue, may proliferate as stem cells, and/or may bind to skeletal muscle tissue, differentiate into functional myoblasts cells, and subsequently develop into functioning myofibers.

The terms “cell surface antigen” and “cell surface marker” as used herein may be any antigenic structure on the surface of a cell. The cell surface antigen may be, but is not limited to, a tumor-associated antigen, a growth factor receptor, a viral-encoded surface-expressed antigen, an antigen encoded by an oncogene product, a surface epitope, a membrane protein which mediates a classical or atypical multi-drug resistance, an antigen which mediates a tumorigenic phenotype, an antigen which mediates a metastatic phenotype, an antigen which suppresses a tumorigenic phenotype, an antigen which suppresses a metastatic phenotype, an antigen which is recognized by a specific immunological effector cell such as a T-cell, and an antigen that is recognized by a non-specific immunological effector cell such as a macrophage cell or a natural killer cell. Examples of “cell surface antigens” include, but are not limited to, CD3, CD4, CD8, CD34, CD90 (Thy-1) antigen, CD117, CD38, CD56, CD61, CD41, glycophorin A and HLA-DR, AC133 defining a subset of CD34⁺ cells, CD19, and HLA-DR. Cell surface molecules may also include carbohydrates, proteins, lipoproteins or any other molecules or combinations thereof, that may be detected by selectively binding to a ligand or labeled molecule and by methods such as, but not limited to, flow cytometry.

The term “cell surface indicator” as used herein refers to a compound or a plurality of compounds that will bind to a cell surface antigen directly or indirectly and thereby selectively indicate the presence of the cell surface antigen. Suitable “cell surface indicators” include, but are not limited to, cell surface antigen-specific monoclonal or polyclonal antibodies, or derivatives or combinations thereof, and which may be directly or indirectly linked to a signaling moiety. The “cell surface indicator” may be a ligand that can bind to the cell surface antigen, wherein the ligand may be a protein, peptide, carbohydrate, lipid or nucleic acid that is directly or indirectly linked to a signaling moiety.

By “detectably labeled” is meant that a polypeptide or a fragment thereof substituted with a fluorophore, or that is substituted with some other molecular species that elicits a physical or chemical response that can be observed or detected by the naked eye or by means of instrumentation such as, without limitation, scintillation counters, calorimeters, UV spectrophotometers and the like. As used herein, a “label” or “tag” refers to a molecule that, when appended by, for example, without limitation, covalent bonding or hybridization, to another molecule, for example, also without limitation, a polynucleotide or polynucleotide fragment, provides or enhances a means of detecting the other molecule. A fluorescence or fluorescent label or tag emits detectable light at a particular wavelength when excited at a different wavelength. A radiolabel or radioactive tag emits radioactive particles detectable with an instrument such as, without limitation, a scintillation counter. Other signal generation detection methods include: chemiluminescence, electrochemiluminescence, raman, calorimetric, hybridization protection assay, and mass spectrometry. Particularly useful in the methods of the present disclosure are reporter polypeptides that are encoded by genetic elements incorporated in the genome of the putative stem cells. Accordingly, such cells will not dilute out the reporter due to proliferation of the cells-each progeny cell will have the expressed reporter and the intensity of the reporter signal, such as bioluminescence, will have defined relationship to the number of cells.

The term “DNA amplification” as used herein refers to any process that increases the number of copies of a specific DNA sequence by enzymatically amplifying the nucleic acid sequence. A variety of processes are known. One of the most commonly used is the polymerase chain reaction (PCR), which is defined and described in later sections below. The PCR process of Mullis is described in U.S. Pat. Nos. 4,683,195 and 4,683,202. PCR involves the use of a thermostable DNA polymerase, known sequences as primers, and heating cycles, which separate the replicating deoxyribonucleic acid (DNA), strands and exponentially amplify a gene of interest. Any type of PCR, such as quantitative PCR, RT-PCR, hot start PCR, LAPCR, multiplex PCR, touchdown PCR, etc., may be used. Advantageously, real-time PCR is used. In general, the PCR amplification process involves an enzymatic chain reaction for preparing exponential quantities of a specific nucleic acid sequence. It requires a small amount of a sequence to initiate the chain reaction and oligonucleotide primers that will hybridize to the sequence. In PCR the primers are annealed to denatured nucleic acid followed by extension with an inducing agent (enzyme) and nucleotides. This results in newly synthesized extension products. Since these newly synthesized sequences become templates for the primers, repeated cycles of denaturing, primer annealing, and extension results in exponential accumulation of the specific sequence being amplified. The extension product of the chain reaction will be a discrete nucleic acid duplex with a termini corresponding to the ends of the specific primers employed.

The term “polymerase chain reaction” or “PCR” as used herein refers to a thermocyclic, polymerase-mediated, DNA amplification reaction. A PCR typically includes template molecules, oligonucleotide primers complementary to each strand of the template molecules, a thermostable DNA polymerase, and deoxyribonucleotides, and involves three distinct processes that are multiply repeated to effect the amplification of the original nucleic acid. The three processes (denaturation, hybridization, and primer extension) are often performed at distinct temperatures, and in distinct temporal steps. In many embodiments, however, the hybridization and primer extension processes can be performed concurrently. The nucleotide sample to be analyzed may be PCR amplification products provided using the rapid cycling techniques described in U.S. Pat. Nos. 6,569,672; 6,569,627; 6,562,298; 6,556,940; 6,569,672; 6,569,627; 6,562,298; 6,556,940; 6,489,112; 6,482,615; 6,472,156; 6,413,766; 6,387,621; 6,300,124; 6,270,723; 6,245,514; 6,232,079; 6,228,634; 6,218,193; 6,210,882; 6,197,520; 6,174,670; 6,132,996; 6,126,899; 6,124,138; 6,074,868; 6,036,923; 5,985,651; 5,958,763; 5,942,432; 5,935,522; 5,897,842; 5,882,918; 5,840,573; 5,795,784; 5,795,547; 5,785,926; 5,783,439; 5,736,106; 5,720,923; 5,720,406; 5,675,700; 5,616,301; 5,576,218 and 5,455,175, the disclosures of which are incorporated by reference in their entireties. Other methods of amplification include, without limitation, NASBR, SDA, 3SR, TSA and rolling circle replication. It is understood that, in any method for producing a polynucleotide containing given modified nucleotides, one or several polymerases or amplification methods may be used. The selection of optimal polymerization conditions depends on the application.

The term “directly delivering” as used herein refers to delivering a pharmaceutically acceptable agent or preparation, or a suspension of isolated or cultured cells, into a mass of target cells or population of cells within a defined location within a subject mammal, whereby the preparation is not delivered by administration into the circulatory system to be distributed throughout the body rather than specifically or mainly to the target tissue. It is expected that the administration may be by injection near the target tissue or into a vessel leading into the area to be treated.

The term “expressed” or “expression” as used herein refers to transcription from a gene to give an RNA nucleic acid molecule at least complementary in part to a region of one of the two nucleic acid strands of the gene. The term “expressed” or “expression” as used herein also refers to the translation from said RNA nucleic acid molecule to give a protein, a polypeptide or a portion thereof.

The term “flow cytometer” as used herein refers to any device that will irradiate a particle suspended in a fluid medium with light at a first wavelength, and is capable of detecting a light at the same or a different wavelength, wherein the detected light indicates the presence of a cell or an indicator thereon. The “flow cytometer” may be coupled to a cell sorter that is capable of isolating the particle or cell from other particles or cells not emitting the second light The term “genome” as used herein refers to all the genetic material in the chromosomes of a particular organism. Its size is generally given as its total number of base pairs. Within the genome, the term “gene” refers to an ordered sequence of nucleotides located in a particular position on a particular chromosome that encodes a specific functional product (e.g., a protein or RNA molecule).

The terms “heterologous”, “exogenous” and “foreign” are used interchangeably herein and in general refer to a biomolecule such as a nucleic acid or a protein that is not normally found in a certain organism or in a certain cell, tissue or other component contained in or produced by an organism.

The term “isolated” as used herein may refer to a nucleic acid or polypeptide separated from at least one other component (e.g., nucleic acid or polypeptide) present with the nucleic acid or polypeptide in its natural source. In one embodiment, the nucleic acid or polypeptide is found in the presence of (if anything) only a solvent, buffer, ion, or other components normally present in a solution of the same. The terms “isolated” and “purified” do not encompass nucleic acids or polypeptides present in their natural source. The term “isolated” as used herein may also refer to a cell or population of cells removed from its/their natural environment such as a donor animal or tissue thereof, or removed from recognizably differing cells isolated from a subject mammal or tissue thereof.

The term “lentivirus” as used herein refers to a genus of retroviruses that can infect dividing and non-dividing cells. Several examples of lentiviruses include HIV (human immunodeficiency virus; including HIV type 1, and HIV type 2), the etiologic agent of the human acquired immunodeficiency syndrome (AIDS); visna-maedi, which causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which cause immune deficiency and encephalopathy in sub-human primates.

A lentiviral genome is generally organized into a 5′ long terminal repeat (LTR), the gag gene, the pol gene, the env gene, the accessory genes (nef, vif, vpr, vpu) and a 3′ LTR. The viral LTR is divided into three regions called U3, R and U5. The U3 region contains the enhancer and promoter elements. The U5 region contains the polyadenylation signals. The R (repeat) region separates the U3 and U5 regions and transcribed sequences of the R region appear at both the 5′ and 3′ ends of the viral RNA. See, for example, “RNA Viruses: A Practical Approach” Alan J. Cann, Ed., Oxford University Press, (2000); Narayan & Clements. Gen. Virology 70:1617-1639 (1989); Fields et al., Fundamental Virology Raven Press. (1990); Miyoshi et al., J Virol. 72:8150-8157 (1998): U.S. Pat. No. 6,013,516.

The term “notexin” as used herein refers to the presynaptically active, toxic phospholipase A2s, which are principal components of the venom of the Australian tiger snake.

The term “vector” as used herein refers a vehicle into which a genetic element encoding a polypeptide may be operably inserted so as to bring about the expression of that polypeptide. A vector may be used to transform, transduce or transfect a subject mammal cell so as to bring about expression of the genetic element it carries within the subject mammal cell. Examples of vectors include plasmids, cosmids, bacmids, bacteriophages such as lambda phage or M13 phage, and animal viruses such as lentivirus, adenovirus, adeno-associated virus (AAV), cytomegalovirus (CMV), herpes simplex virus (HSV), papillomavirus, retrovirus, and simian virus 40 (SV40). A vector utilized as part of an expression system may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. The viral particle may include one or more proteins that help facilitate assembly of the viral particle, transduction of the subject mammal cell, and transport of the vector polynucleotide sequence within the subject mammal cell, among other functions. The term “lentiviral vector” as used herein refers to a lentiviral vector designed to operably insert an exogenous polynucleotide sequence into a subject mammal genome.

The term “proliferative status” as used herein refers to whether a population of cells, and in particular stem cells or progenitor cells, or a subpopulation thereof, are dividing and thereby increasing in number, in the quiescent state, or whether the cells are not proliferating, dying or undergoing apoptosis.

The terms “modulating the proliferative status” or “modulating the proliferation” as used herein refers to the ability of a compound to alter the proliferation rate of a population of stem cells, including muscle satellite cells, or progenitor cells. A compound may be toxic wherein the proliferation of the cells is slowed or halted, or the proliferation may be enhanced such as, for example, by the addition to the cells of a cytokine or growth factor, thereby increasing the proliferative rate.

The term “non-invasive” as used herein refers to a method of obtaining qualitative or quantitative data, and in particular luminescence, fluorescence or PET measurements or images, and the like, without removing tissue or other biological samples from a subject animal. In general, non-invasive techniques do not include any surgical methods or dissection of the animal or in any way harm the subject. Non-invasive techniques of imaging, for example, may be by methods or apparatus not in physical contact with the animal, such as, but not limited to, a camera-based system, a cooled charge-coupled diode based system and the like. The imaging system may be a combination of systems if the signals relating to more than a single reporter are to be detected.

The terms “oligonucleotide” and “polynucleotide” as used herein generally refer to any polyribonucleotide or polydeoxribonucleotide that may be unmodified RNA or DNA or modified RNA or DNA. Thus, for instance, polynucleotides as used herein refers to, among others, single-and double-stranded DNA, DNA that is a mixture of single-and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The terms “nucleic acid,” “nucleic acid sequence,” or “oligonucleotide” also encompass a polynucleotide as defined above.

The terms “operably linked” or “operatively linked” refer to the configuration of the coding and control sequences so as to perform the desired function. Thus, control sequences operably linked to a coding sequence are capable of effecting the expression of the coding sequence and regulating in which tissues, at what developmental time points, or in response to which signals, etc., a gene is expressed. A coding sequence is operably linked to or under the control of transcriptional regulatory regions in a cell when DNA polymerase will bind the promoter sequence and transcribe the coding sequence into mRNA that can be translated into the encoded protein. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence. Such intervening sequences include but are not limited to enhancer sequences which are not transcribed or are not bound by polymerase.

The term “polynucleotide” as used herein refers to DNAs or RNAs as defined above that may contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

The terms “polypeptide” or “protein” as used herein refer to encompass a protein, a glycoprotein, a polypeptide, a peptide, and the like, whether isolated from nature, of viral, bacterial, plant, or animal (e.g., mammalian, such as human) origin, or synthetic, and fragments thereof.

The term “promoter” as used herein refers to a DNA sequence that determines the site of transcription initiation by an RNA polymerase. A “promoter-proximal element” may be a regulatory sequence within about 200 base pairs of the transcription start site. Useful promoters also include exogenously inducible promoters. These are promoters that can be “turned on” in response to an exogenously supplied agent or stimulus, which is generally not an endogenous metabolite or cytokine.

The term “reporter” or “reporter polypeptide” as used herein refers to a molecule that may be detected, where the reporter is an adjunct to the cell, nucleic acid or polypeptide under study. The reporter provides a signal such as, but not limited to, a bioluminescence discharge, fluorescent activity, radioactive decay particles, an enzyme activity and the like that may be qualitatively or quantitatively related to the activity or amount of the subject under study. The reporter may be, for example, but is not limited to, an enzyme such as a peroxidase, or a luciferase that in the presence of a bioluminescence initiator emits detectable bioluminescence. Suitable luciferases include, but are not limited to, such as firefly luciferase, Renilla luciferase and the like, or mutants or variants thereof. In particular, the reporter is a polypeptide encoded by a nucleic acid and which may be inserted in the genome of a donor animal, whereby the cells of the subject animal, including stem cells thereof, include the heterologous nucleic acid, are capable of expressing the reporter polypeptide, and therefore may be specifically detected.

The term “bioluminescence” as used herein refers to a type of chemiluminescent, emission of light by biological molecules, particularly proteins. The essential condition for bioluminescence is molecular oxygen, either bound or free in the presence of an oxygenase, a luciferase, which acts on a substrate, a luciferin in the presence of molecular oxygen and transforms the substrate to an excited state, which upon return to a lower energy level releases the energy in the form of light.

The term “luciferase” as used herein refers to oxygenases that catalyze a light emitting reaction. For instance, bacterial luciferases catalyze the oxidation of flavin mononucleotide and aliphatic aldehydes, which reaction produces light. Another class of luciferases, found among marine arthropods, catalyzes the oxidation of cypridina luciferin, and another class of luciferases catalyzes the oxidation of coleoptera luciferin. Thus, “luciferase” refers to an enzyme or photoprotein that catalyzes a bioluminescent reaction. The luciferases such as firefly and Renilla luciferases are enzymes that act catalytically and are unchanged during the bioluminescence generating reaction. The luciferase photoproteins, such as the aequorin and obelin photoproteins to which luciferin is non-covalently bound, are changed by release of the luciferin, during bioluminescence generating reaction. The luciferase is a protein that occurs naturally in an organism or a variant or mutant thereof, such as a variant produced by mutagenesis that has one or more properties, such as thermal or pH stability, that differ from the naturally-occurring protein. Luciferases and modified mutant or variant forms thereof are well known. Reference, for example, to “Renilla luciferase” means an enzyme isolated from member of the genus Renilla or an equivalent molecule obtained from any other source, such as from another Anthozoa, or that has been prepared synthetically.

“Bioluminescent protein” refers to a protein capable of acting on a bioluminescent initiator molecule substrate to generate or emit bioluminescence.

“Fluorescent acceptor molecule” refers to any molecule that can accept energy emitted as a result of the activity of a bioluminescent donor protein, and re-emit it as light energy.

The term “bioluminescent initiator molecule” as used herein refers to a molecule that can react with a bioluminescent donor protein to generate bioluminescence. The bioluminescence initiator molecule includes, but is not limited to, coelenterazine, analogs thereof, and functional derivatives thereof. Derivatives of coelenterazine include, but are not limited to, coelenterazine 400a, coelenterazine cp, coelenterazine f, coelenterazine fcp, coelenterazine h, coelenterazine hcp; coelenterazine ip, coelenterazine n, coelenterazine O, coelenterazine c, coelenterazine c, coelenterazine i, coelenterazine icp, coelenterazine 2-methyl, benzyl-coelenterazine bisdeoxycoelenterazine, and deep blue coelenterazine (DBC) (described in more detail in U.S. Pat. Nos. 6,020,192; 5,968,750 and 5,874,304).

In general, coelenterazines are known to luminesce when acted upon by a wide variety of bioluminescent proteins, specifically luciferases. Useful, but non-limiting, coelenterazines are disclosed in U.S. patent application Ser. No. 10/053,482, filed Nov. 2, 2001, the disclosure which is hereby incorporated by reference in its entirety. Coelenterazines are available from Promega Corporation, Madison, Wis. and from Molecular Probes, Inc., Eugene, Oreg. Coelenterazines may also be synthesized as described for example in Shimomura et al., Biochem. J. 261: 913-20, 1989; Inouye et al., Biochem. Biophys. Res. Comm. 233: 349-53, 1997; and Teranishi et al., Anal. Biochem. 249: 37-43, 1997.

The terms “fluorescent dye” and “fluorescent label” as used herein includes all known fluors, including rhodamine dyes (e.g., tetramethylrhodamine, dibenzorhodamine, see, e.g., U.S. Pat. No. 6,051,719); fluorescein dyes; “BODIPY” dyes and equivalents.

The term “caged substrate” as used herein refers to a molecule comprising a “caging group”, which is a moiety that can be employed to reversibly block, inhibit, or interfere with the activity (e.g., the biological activity) of the molecule such as, but not limited to, a polypeptide, a nucleic acid, a small molecule, a drug, etc.). The caging groups can, e.g., physically trap an active molecule inside a framework formed by the caging groups. Typically, however, one or more caging groups are associated (covalently or non-covalently) with the molecule but do not necessarily surround the molecule in a physical cage. For example, a single caging group covalently attached to an amino acid side chain required for the catalytic activity of an enzyme can block the activity of the enzyme. The enzyme would thus be caged even though not physically surrounded by the caging group. As another example, covalent attachment of a single caging group to an amino acid side chain that is phosphorylated by a kinase in a kinase substrate can block phosphorylation of that substrate by the kinase. Caging groups can be, e.g., relatively small moieties such as carboxyl nitrobenzyl, 2-nitrobenzyl, nitroindoline, hydroxyphenacyl, DMNPE, or the like, or they can be large bulky moieties such as a protein or a bead. Caging groups can be removed from a molecule, or their interference with the molecule's activity can be otherwise reversed or reduced, by exposure to an appropriate type of uncaging energy and/or exposure to an uncaging chemical, enzyme, or the like.

The terms “test compound” and “candidate compound” as used herein refer to any chemical entity, pharmaceutical, drug, and the like that is a candidate for use to treat or prevent a disease, illness, sickness, or disorder of bodily function (e.g., cancer), or merely intended to have an effect on a test subject cell or cell line or engrafted cell population. Test compounds may comprise, but are not limited to, both known and potential therapeutic compounds. A test compound can be determined to be therapeutic by screening using the screening methods of the present disclosure.

The term “tissue” as used herein refers to a group or collection of similar cells and their intercellular matrix that act together in the performance of a particular function. The primary tissues are epithelial, connective (including blood), skeletal, muscular, glandular and nervous.

The term “transgene” as used herein refers to a nucleic acid sequence encoding, for example, a reporter polypeptide that is partly or entirely heterologous, i.e., foreign, to the transgenic animal or cell into which it is introduced.

The term “transgenic animal” as used herein refers to a non-human animal, such as a mouse, in which cells of the animal contain a heterologous nucleic acid introduced by way of human intervention, such as by transgenic techniques well known in the art. The nucleic acid is introduced into a cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animal, the transgene causes cells to express a recombinant form of the subject polypeptide, e.g. either agonistic or antagonistic forms, or in which the gene has been disrupted. In certain embodiments, the genome of the animal has been modified such that a heterologous gene expression element is inserted so as to be operably linked to an endogenous coding sequence.

### Discussion

The methods of the present disclosure have allowed the isolation of a subset of muscle satellite cells that have the properties associated with stem cells. In particular, the subject subpopulation of satellite cells was isolated from the heterogenous population of satellite cells using a specific combination of cell markers to identify and partition the subset, and show that the subset contained muscle stem cells based on the classic single cell functional definition.

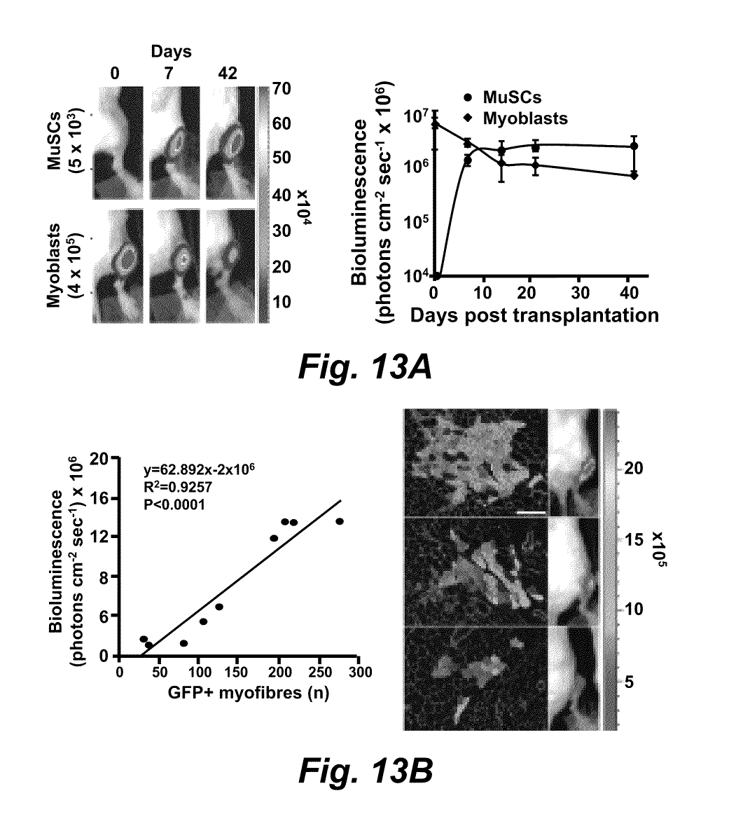

The engrafting of a single cell that had been isolated from a population of satellite cells according to the methods of the disclosure, and as shown in FIGS. 13A and 13B for example, showed that the implanted single cell proliferates to a detectable level in the tissue and that the cell was a true muscle stem cell. This result contrasted with previous studies that have not been able to demonstrate that a specific subpopulation of heterologous satellite cells harbors true muscle stem cells that self-renew and differentiate (the classic stem cell definition derived from the hematopoietic stem cell field, i.e. a single stem cell gives rise to copies of itself and to more specialized cells that contribute to tissue regeneration).

To detect a single stem cell and validate its properties, a non-invasive method for imaging according to the disclosure, analyzes only those mice with an adequate signal (i.e. enough cells) and at the right time, thereby avoiding sacrificing mice so that they are unavailable for further analysis). For a liquid tissue such as blood, taking samples over time from a living animal, with the representative of the whole tissue and do not require sacrifice of the animal, is possible. For a solid tissue, however, this is not possible. Extended time periods of monitoring implanted cell proliferation in a single animal without sacrificing is desirable, for example, to determine if there is undesirable and uncontrolled proliferation of the implanted cells to form tumors.

The ability to monitor stem cells and their function in vivo is currently restricted to static histological images that provide a snapshot of the degree of participation of the cells in a given tissue at a given time. Using such classical histological methods, the contribution of the stem cells to adult tissues is difficult to quantify, preventing efficiency comparisons between different putative stem cells, or methods of stem cell delivery. In addition, analyses of stem cell contributions to solid tissues are cumbersome and expensive, requiring numerous mice, as for each time point the sacrifice of several animals is necessary.

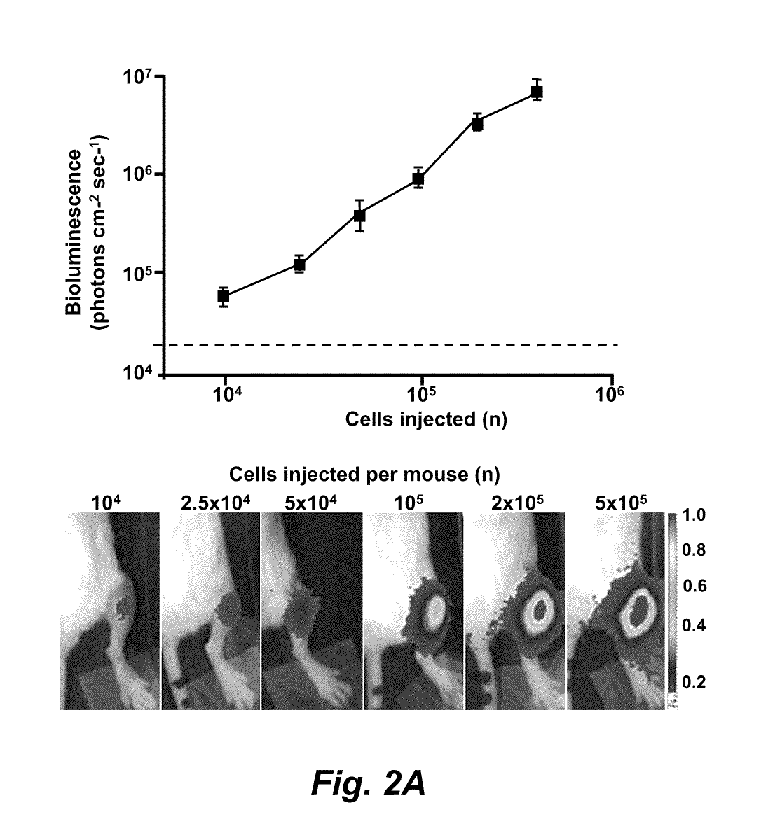

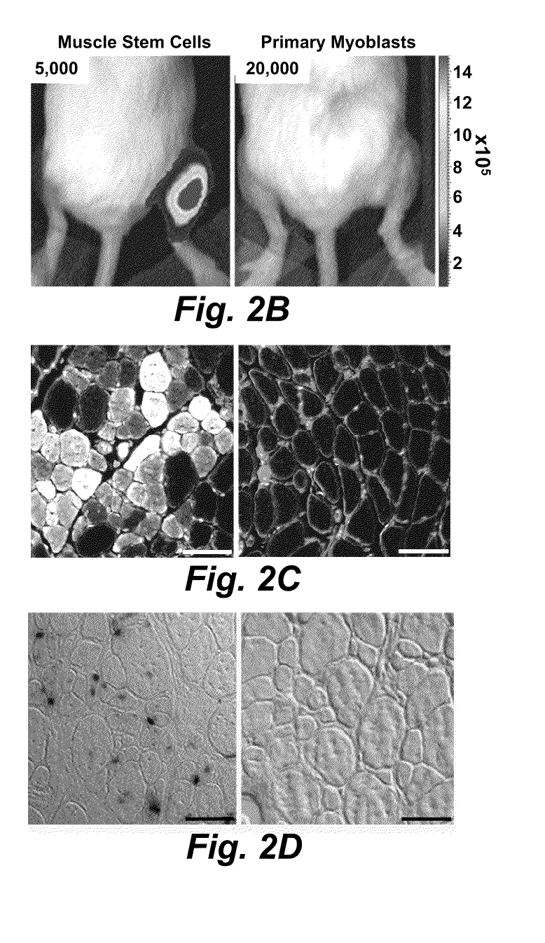

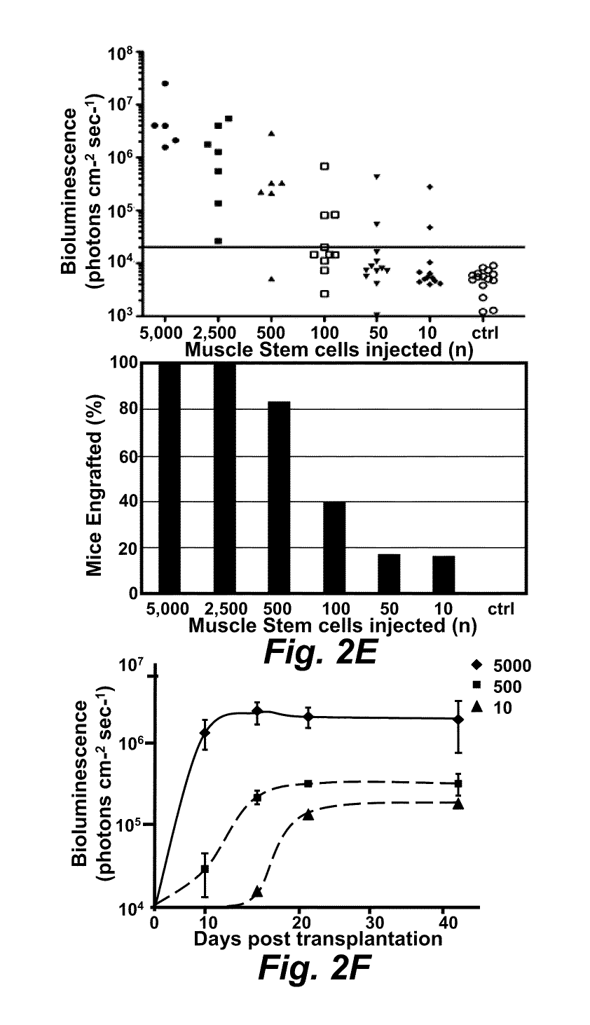

The embodiments of the present disclosure provide methods for monitoring stem cell proliferation in solid tissues in a dynamic and non-invasive manner in living animals, not possible using classical histological analyses (as illustrated, for example in FIGS. 2A-2F, and 13A and 13B). The kinetics and magnitude of stem cell contributions to tissues can be monitored repeatedly in the same animal. Moreover, in addition to proliferative behavior and expansion of the stem cell progeny, the length of time necessary to reach a steady state, or homeostasis, is readily revealed. In addition, the engraftment of transplanted stem cells within the stem cell compartment, and their potential to mount a proliferative response upon tissue injury, can be evaluated repeatedly over an extended period of time.

The methods of the disclosure may be used to monitor stem cell fate over an extended period in a single cell recipient animal in a non-invasive manner by making use of, but not limited to, bioluminescence imaging of a reporter activity. While not wishing to be limiting, one reporter activity suitable for use in the methods of the disclosure is luciferase-generated bioluminescence. It is contemplated, however, that other imaging systems such as fluorescence detection or PET imaging may be used individually or in combination. For example, a vector and transgenic mice capable of expressing a combination of reporters that may be used in the embodiments of the methods of the disclosure is described in U.S. patent Application Serial No. 200660277613, entitled “Multimodality Imaging of Reporter Gene Expression using a Fusion Vector in Living Cells and Animals”, incorporated herein by reference in its entirety.

For example, adult stem cells can be isolated from transgenic mice engineered to ubiquitously and constitutively express luciferase. Alternatively, it is contemplated that stem cells can be genetically engineered, immediately following isolation, with such as, but not limited to, a lentiviral vector encoding and expressing luciferase. This technology can be readily extended so that the lentiviral vectors or transgenic mice drive the expression of luciferase, β-galactosidase or β-lactamase under the control of diverse promoters that are characteristic of quiescence, activation, and differentiation of the stem cells and could therefore serve as readouts for these different stem cell states. These reporters can then be analyzed simultaneously in living mice using caged substrates, for example Luc-Galactosidase in the case of β-galactosidase. It is further contemplated that the reporter activity may be placed under the expression control of a promoter specific to a the type of stem cell under investigation, such as, but not limited to, Myf5 muscle-specific promoter, or an inducible promoter that may respond to an exogenous inducer delivered to the recipient engrafted subject mammal, or an endogenous inducer such as a cytokine, growth factor or the like.

The methods of the disclosure provide many applications suitable for use in stem cell biology studies. The methods of the disclosure may also extend to solid tissues those studies that previously were only possible with stem cells in liquid tissues such as blood, that can be readily sampled without sacrifice of the animal and for which a small sample is representative of the whole tissue. The methods of the disclosure further allow diverse cell populations to be directly compared in diverse animal models of injury or genetic disease. In addition, to validate that a protein or small molecule (drug) is efficacious in potentiating stem cell self-renewal and expansion to yield adequate numbers of stem cells while retaining their stem cell state (regenerative properties), a means of monitoring the dynamic behavior of the stem cells following transplantation into mice, as provided by this technology, is required. In particular, the potential to test the effects of a drug or potential therapeutic agent on cultured stem cells would enable a determination of whether the cell that is propagated is truly a stem cell, i.e., the progeny comprise both more stem cells and cells that differentiate to form the tissue of interest. The embodiments of the methods of disclosure are useful for stem cell validation by transplantation of stem cells as single cells and for expansion of stem cells to high numbers for clinical use. For example, using the methods of the disclosure, it has been shown that a particular subset of cells from a population of muscle satellite cells have the properties associated with a true stem cell, especially of expansion of the population of cells from a single engrafted cell of the isolated subset.

In embodiments of the methods of the disclosure, therefore, cell number may be determined as a function of luciferase activity during the proliferative phase, the achievement of homeostasis (plateau), and in response to serial tissue injury. The measurements may, for example, provide data regarding muscle stem cell behavior in a dynamic manner, revealing the kinetics and magnitude of the stem cell response over time.

Moreover, the experiments using these methods can allow a direct comparison in the same animals of stem cells such as, for example, muscle stem cells and their more specialized progenitors, myoblasts, in vivo. The regenerative potential of committed myoblasts (muscle stem cells grown in tissue culture) compared with that in observed freshly isolated muscle stem cells (muscle stem cells never exposed to tissue culture) was markedly different. Myoblasts do not increase in numbers following transplantation into muscles, or in response to tissue damage, by contrast with muscle stem cells which exhibited 100-fold changes in cell numbers.

To validate this strategy as a quantitative assay of muscle stem cell proliferation in vivo, bioluminescence images of increasing numbers of cells both in vitro and in vivo were first acquired. For this purpose, populations of luciferase⁺-muscle stem cells were isolated by flow cytometry using antibodies that specifically selected for CD34⁺, α7-integrin⁺, Sca-1⁻, CD45⁻, CD11b⁻, CD31⁻ cells, a subset of muscle satellite cells having extensive stem cell activity and regenerative capacity. These experiments revealed the time-course and the magnitude of the proliferative response of which adult muscle stem cells and their progeny are capable in vivo. Luciferase activity assayed in protein extracts from proliferating myoblasts and differentiated myofibers confirmed that the luciferase activity was not affected by the stage of muscle differentiation, validating the assay as a quantitative measure of cell numbers and data regarding proliferation.

The methods of the disclosure also allowed single muscle stem cell transplants to be evaluated, as it enabled rapid screening of recipient mice to identify successfully engrafted animals (mice in which a single engrafted stem cell had proliferated to at least 10,000 cells, the threshold of detection). Without a method for non-invasive imaging, the evaluation of single cell transplanted mice would have been extremely labor intensive and imprecise, requiring numerous mice sacrificed at different time points and analyzed blindly using classical histological approaches. The imaging technology indicated which mice and at what time these mice were worthy of further consideration, and providing evidence that the adult muscle stem cells isolated using specific cell type markers were true stem cells with both self-renewal and differentiation capacity, and comparison of adult muscle stem cells with myoblasts (the more committed derivatives of muscle stem cells) revealed that only muscle stem cells are able to maintain their self-renewal capacity in vivo.

The non-invasive methods of the present disclosure, therefore, allow direct comparisons of the quantitative and dynamic properties of diverse stem cell populations, mouse models, injury paradigms and therapeutic agents, not only for muscle but all transplanted stem cell types.

Embodiments of the present disclosure, therefore, provide methods for qualitatively and/or quantitatively detecting in vivo a population of engrafted stem cells in a tissue of a subject mammal. By non-invasively monitoring the presence and/or the amount of a population of engrafted stem cells in a subject mammal, it is possible to perform time-dependent observations using a single subject mammal rather than requiring the periodic sacrifice of multiple (and statistically significant numbers of) engrafted animals. The methods of the present disclosure comprise obtaining a population of primary stem cells, advantageously derived from a transgenic donor wherein each cell includes a heterologous nucleic acid encoding a reporter polypeptide. The heterologous nucleic acid is preferably operably linked to a promoter region that leads to the constitutive expression of the reporter, or may be an inducible promoter that allows expression of the heterologous nucleic acid in the presence of an appropriate inducer at the selected time. The isolated stem cell population may be delivered directly to a tissue of the subject mammal, or systemically administered, whereupon some or all of the delivered stem cells may migrate to a desired solid tissue.

The tissue-engrafted stem cells may then be detected by monitoring the presence of the reporter activity in the tissues of the subject mammal. The detection method for use in the methods of the disclosure may be selected so as to be non-invasive and, therefore, not require dissection and removal of the tissue under study from the subject mammal. For example, an advantageous reporter/detection method system that is non-invasive is a luciferase generation of bioluminescence that can be detected by whole body detection of the emitted light using CCD's and image recording apparatus. By selection of the appropriate wavelength of the bioluminescence it is possible to detect the emission and hence of the stem cells themselves in tissues immediately underlying the skin of the subject mammal.

Proliferation of the stem cells results in amplification of the bioluminescent signal since each of the stem cells, originating from a transgenic subject mammal, has the nucleic acid expressing the reporter integrated into the genome of each cell. A correlation can be shown between the intensity of the bioluminescence emitted and the number of stem cells in the tissues. This information allows the researcher to track the stem cells in the subject mammal body, and migration to and into a solid tissue. It may also allow studies into the proliferation of the stem cells and their modification or commitment to other cell types in response to stimuli such as, but not limited to, tissue disease, death or injury.

It is contemplated that in embodiments of the disclosure, stem cells may be isolated from different transgenic donors, each donor type capable of expressing a different reporter. The stem cells from different tissues, each from a different donor, may then be combined or delivered separately to a subject mammal, and each stem cell type individually monitored.

Other embodiments of the disclosure provide a means to determine the effects of pharmaceutically acceptable agents of the proliferative status of a population of stem cells delivered to a subject mammal. By monitoring the intensity of a bioluminescence signal emitted by engrafted stem cells both before and after the administration of the agent to the subject mammal it is anticipated that a determination may be made as to whether the agent modulates the proliferation of the target stem cells by increasing or decreasing the rate of stem cell proliferation. In other embodiments, the effect of a pharmaceutically acceptable agent on the migration of a population of engrafted stem cells in a subject mammal may be monitored non-invasively and, therefore, over an extended period of time in one subject mammal, rather than in a series of subject mammals, where each is sacrificed at individual time points.

The embodiments of the present disclosure, therefore, encompass methods for in vivo bioluminescence imaging that allow the dynamics of satellite cell behavior to be followed in a manner not possible using conventional retrospective static histological analyses. By imaging luciferase activity, for example, real time quantitative and kinetic analyses can show that donor-derived muscle satellite cells may proliferate and engraft rapidly after injection until homeostasis is reached. Upon injury, donor-derived mononucleated cells rapidly generate massive waves of cell proliferation.

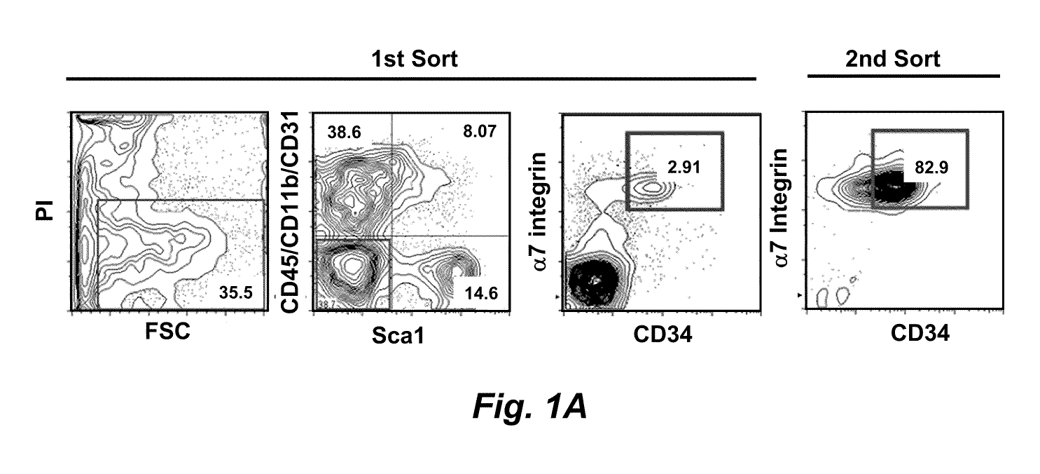

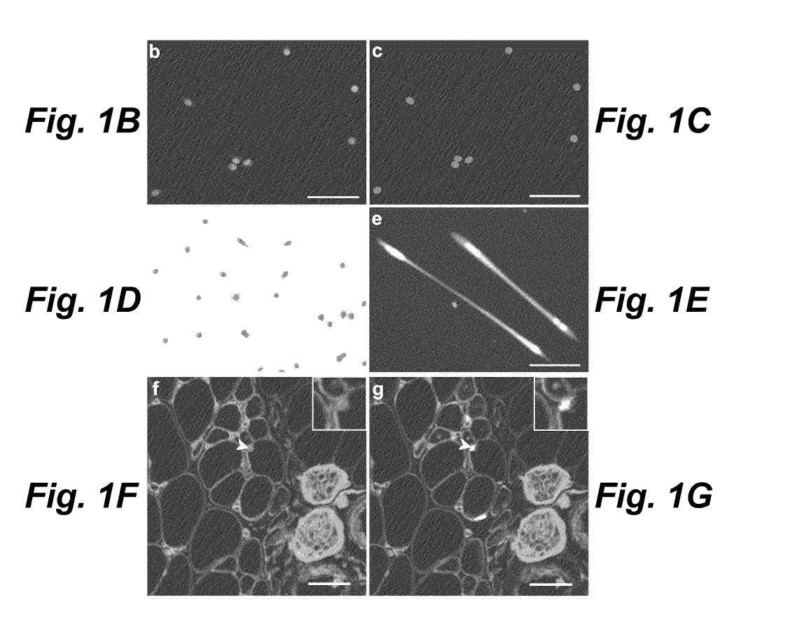

It is anticipated that a stem cell population may be isolated from a transgenic donor by any method that allows for the selection of a population of cells. For example, after magnetic depletion of the population of cells bearing the markers CD45, CD11b, Sca1 and CD31, a combination of the endogenous markers CD34 and α7integrin may be targeted to allow enrichment for a muscle satellite cell population of morphologically round cells that uniformly express the satellite cell-specific transcription factor Pax7, as shown in FIGS. 1A-1C.

When isolated from Myf5-nLacZ transgenic mice and plated in vitro, these cells were activated to express the transcription factor Myf5 (see Tajbakhsh et al., Dev. Dyn. 206: 291 (1996)), evident as β-galactosidase (β-gal) activity, as shown in FIG. 1D, and differentiated to form multinucleated myotubes (FIG. 1E). This satellite cell population was transplanted into Tibialis Anterior (TA) muscles of immunodeficient NOD/SCID mice depleted of endogenous satellite cells by 18Gy irradiation, according to standard procedures of Wakeford et al., Muscle Nerve 14:42 (1991) and Heslop et al., J. Cell. Sci. 113: 2299 (2000), incorporated herein by reference in their entireties.

Four weeks after transplantation, mouse muscles were damaged with notexin (NTX) (see Harris & Johnson, Clin. Exp. Pharmacol. Physiol. 5: 587 (1978); Doyonnas et al., Proc. Natl. Acad. Sci. USA 101: 13507(2004); Sacco et al., J. Cell Biol. 171, 483 (2005) incorporated herein by reference in their entireties) and Myf5⁺ (β-galactosidase⁺) donor-derived cells were subsequently detected in the anatomically defined satellite cell position underneath the basal lamina of myofibers, as illustrated in FIGS. 1A-1F. Classical histological analysis demonstrated that this population of freshly isolated cells homed to the satellite cell niche and responded appropriately to muscle damage by up-regulating expression of the Myf5 transcription factor.

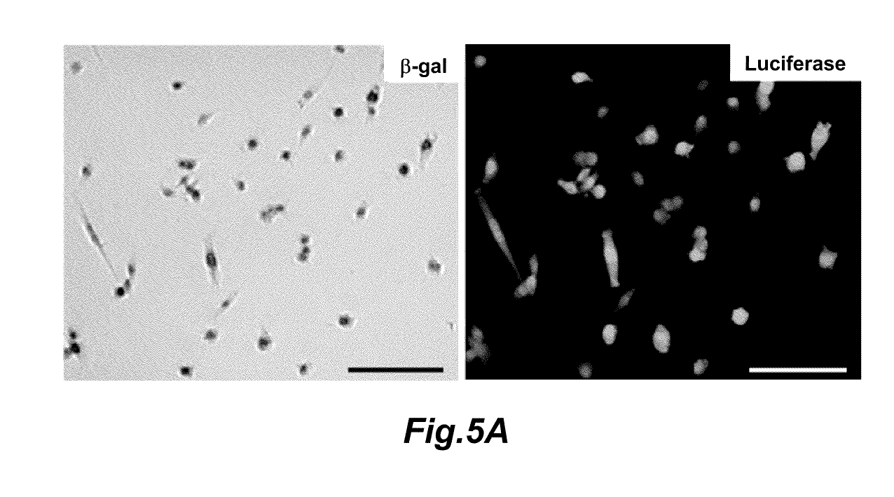

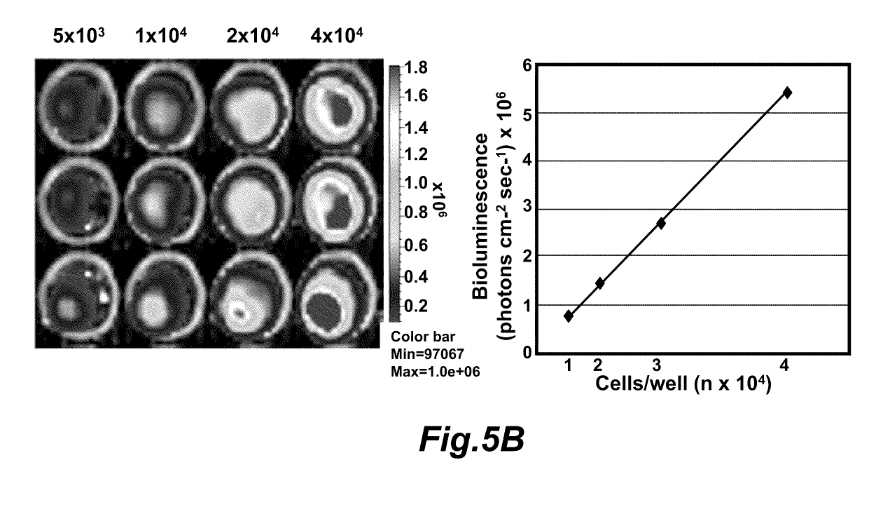

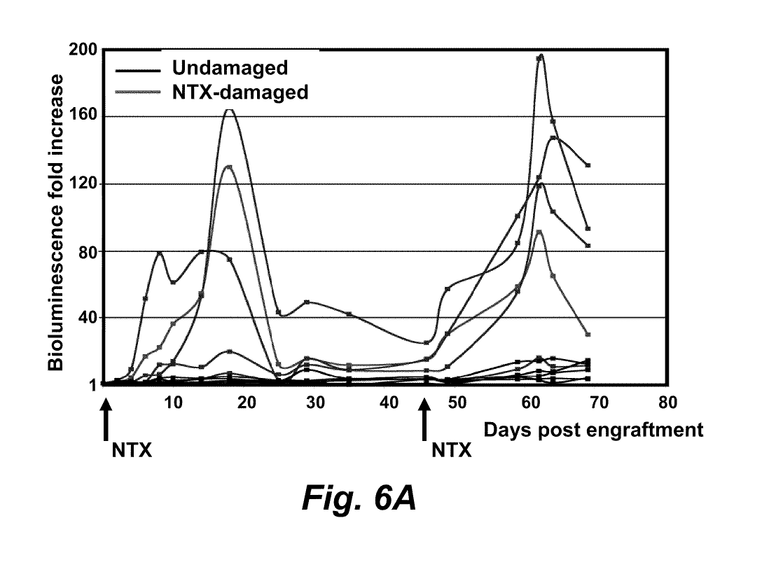

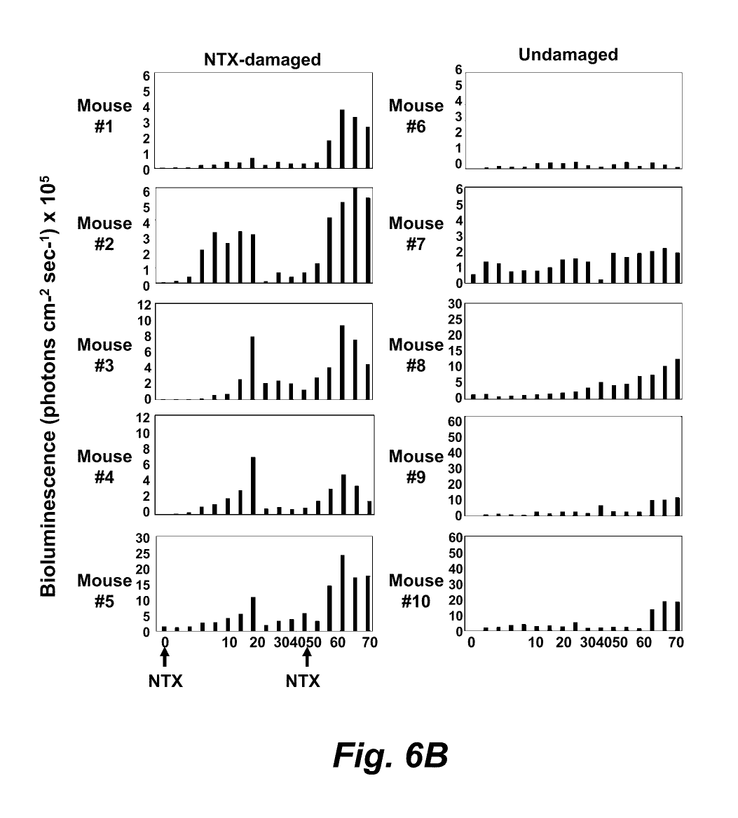

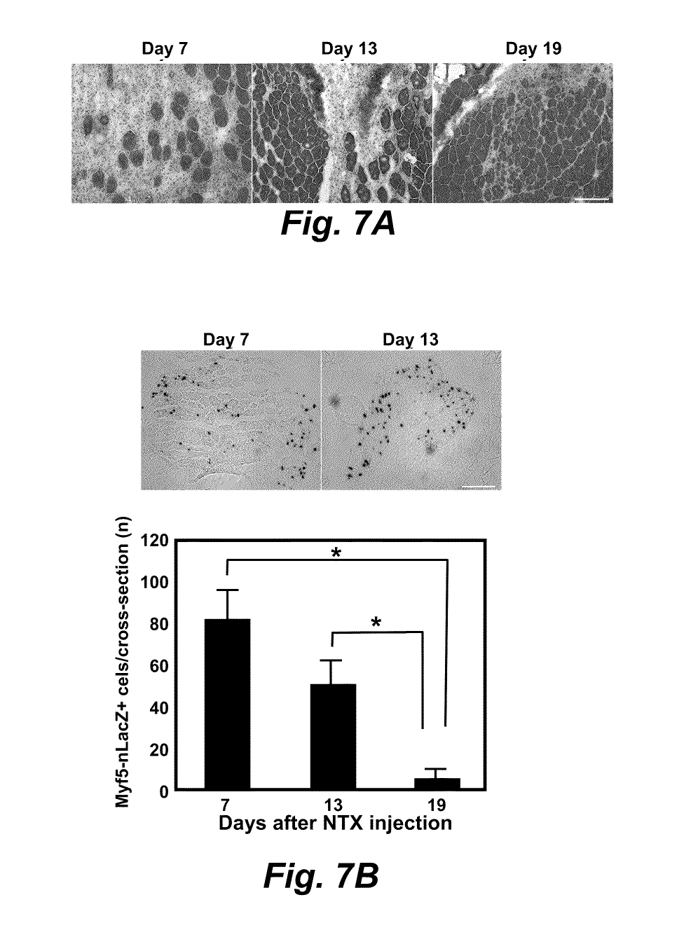

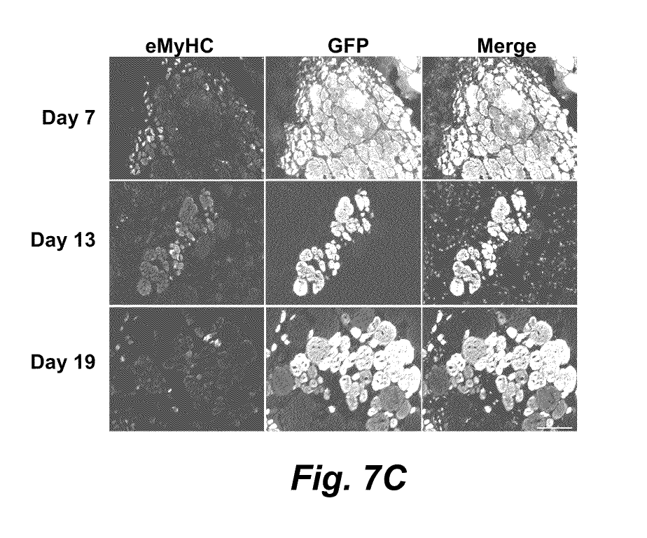

The dynamic assay methods of the present disclosure can complement histological analyses, by providing insights into the kinetics and extent of proliferation of transplanted satellite cells. A sensitive non-invasive bioluminescence imaging assay was developed to monitor satellite cells by first mating Myf5-nLacZ mice with Fluc mice, as previously described (Wehrman et al., Nat. Methods 3, 295 (2006) incorporated herein by reference in its entirety). In these studies, cell number was assessed as the bioluminescence signal derived from constitutive luciferase activity and the activity of the Myf5 promoter was assayed histologically as β-galactosidase activity. The linearity, sensitivity, and reproducibility of the bioluminescence assay for quantifying cell numbers was validated in vitro (FIG. 5B) and in vivo (FIG. 2A). The average luminescent signal/cell detected was 13±3 photons cm⁻²sec⁻¹/cell in vivo, with a minimum number of 10,000 cells detectable above control uninjected legs (FIGS. 2A and 2E).

To validate bioluminescence imaging as an assay for in vivo muscle stem cell function, freshly isolated uncultured satellite cells were compared with cultured primary myoblasts, as these two cell types differ markedly (Montarras et al., Science 309, 2064 (2005)). About 5,000 freshly isolated satellite cells, or a four-fold excess of 20,000 myoblasts, both isolated from the Myf5-nLacZ/Fluc double transgenic mice, were injected into irradiated legs of NOD/SCID recipient mice. FIG. 2B shows a representative example in which, four weeks after transplantation, myoblasts were barely detectable (0.2±0.01×10⁵ photons cm² sec⁻¹) indicating that their numbers had declined. Satellite cells yielded robust luciferase activity (29.0±7.0×10⁵ photons cm⁻² sec⁻¹), a signal corresponding to approximately 3×10⁵ cells, approximating a 60-fold expansion (about 6 doublings).

Histological analysis of injected muscles showed luciferase⁺ myofibers in muscles of mice that received satellite cells, but not myoblasts (FIG. 2C). The histochemistry of NTX damaged muscles revealed the presence of Myf5-nLacZ⁺ cells, indicative of activated satellite cells, following injection of uncultured satellite cells, but not myoblasts (FIG. 2D).

These results confirm that satellite cells, but not myoblasts, successfully engraft, proliferate and give rise to committed progenitors and myoblasts that contribute to muscle fibers. Myoblasts are inefficient in regenerating muscle. However, as death post-transplantation was extensive and the cells that survive did not disperse, but instead fused to myofibers and remained localized at the site of injection (Mouly et al., Acta Myol. 24: 128 (2005); Arcila et al., J. Neurobiol. 33: 185(1997); Rando & Blau, J. Cell Biol. 125: 1275 (1994); Rando & Blau, Methods Cell Biol. 52: 261 (1997); Gussoni et al., Nat. Med. 3: 970 (1997); Gussoni et al., Nature 356: 435 (1992)), the magnitude of the difference in behavior exhibited by the progeny of satellite cells and myoblasts shown here by bioluminescence imaging could not be fully appreciated in previous histological analyses.

To determine the degree of enrichment for cells with engraftment potential in the satellite cell population and the magnitude of the response per engrafted cell, a range of numbers of freshly isolated cells were transplanted into irradiated recipient TA muscles. Bioluminescence was assayed four weeks after transplantation and successful engraftment was defined as persistence of a signal >20,000 photonS/Cm⁻²sec⁻¹, significantly above the background signal detected in control uninjected legs, as illustrated in FIG. 2E.

More than 80% of mice exhibited engraftment when high numbers of satellite cells (500-5,000) were transplanted; but even when as few as 10 cells were transplanted, 16% ( 2/12 mice) exhibited engraftment (FIG. 2E). Notably, the signal plateaued in all cases, equivalent to 145,000 cells (about 5 doublings) for 5,000 cells injected, 39,000 cells for 500 cells injected (about 6 doublings), and 31000 cells for 10 cells injected (about 11 doublings), but the plateau occurred earlier and at a higher level when more cells were injected (FIG. 2F). During the first few weeks following transplantation, a first wave of expansion was observed (FIG. 2F), yielding a plateau indicative of successful persistent cell engraftment.

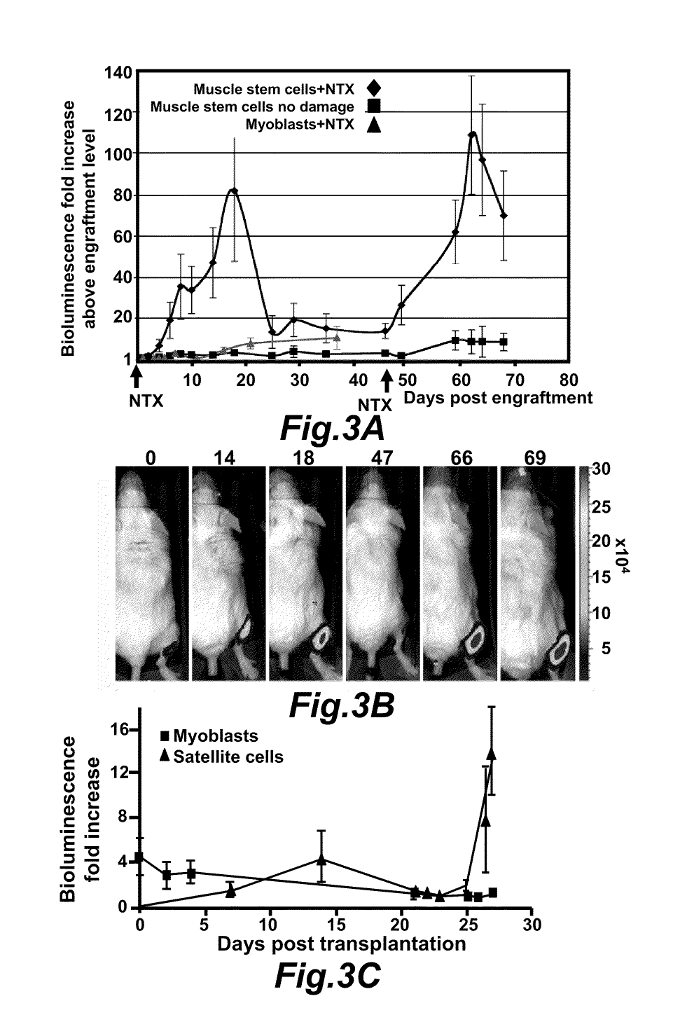

A functional property of adult stem cells is the ability to repeatedly respond to tissue injury by giving rise to substantial numbers of proliferative progenitors. Using bioluminescence imaging by the methods according to the present disclosure, both the kinetics and magnitude of the proliferative response can be determined. Irradiated NOD/SCID recipient mice were injected with 10 or 500 satellite cells, as shown in FIG. 3.

After engrafting of the donor cells, mice were divided into 2 groups, one of which received NTX damage, whereas the other did not. Upon NTX injury, transplanted cells underwent a second wave of about 80-fold expansion, and upon re-injury with NTX a third wave of about 100-fold expansion was observed, assessed as luciferase activity relative to the activity before NTX damage. A peak was observed about 15 days post injury in each case (FIG. 3A). In FIG. 3B, representative bioluminescent images of one of these NTX-damaged mice are shown. This dynamic assay showed a drop in cell number at the end of each regenerative wave of cell expansion, suggesting that cell death may counterbalance stem and progenitor cell proliferation in order to achieve homeostasis post-injury. By contrast, luciferase activity in undamaged control mice remained relatively constant following engraftment (dashed line, FIG. 3A). These results demonstrated that transplanted satellite cells can respond rapidly to serial injury with successive waves of progenitor expansion. The magnitude of the response to two sequential damages suggests that stem cell function persisted over time.

The increase in luciferase signal was indicative of cell number and not due to up-regulation of the luciferase gene or increased access of luciferin to luciferase⁺ cells in a damaged tissue. This was shown by transplanting large numbers of myoblasts (4×10⁵ per muscle), which yielded a detectable bioluminescence signal immediately following injection. By contrast with satellite cells, the bioluminescence signal did not increase, but instead dropped by 50% within 24 hours of injection, in agreement with the post-injection cell death (Mouly et al., Acta Myol. 24, 128 (2005); Fan et al., Muscle Nerve 19, 853 (1996); Barberi et al., Nat. Med. 13, 642 (2007)), and plateaued over the subsequent 4 week period. Moreover, following NTX damage, instead of increasing, the bioluminescent signal did not change, in accordance with the documented inability of myoblasts to proliferate in response to injury (FIG. 3C). These results support that luciferase activity served as a useful readout of cell numbers, facilitating comparisons of satellite cells and myoblasts in a dynamic manner that allows insights into the magnitude and kinetics of their responses to tissue injury.

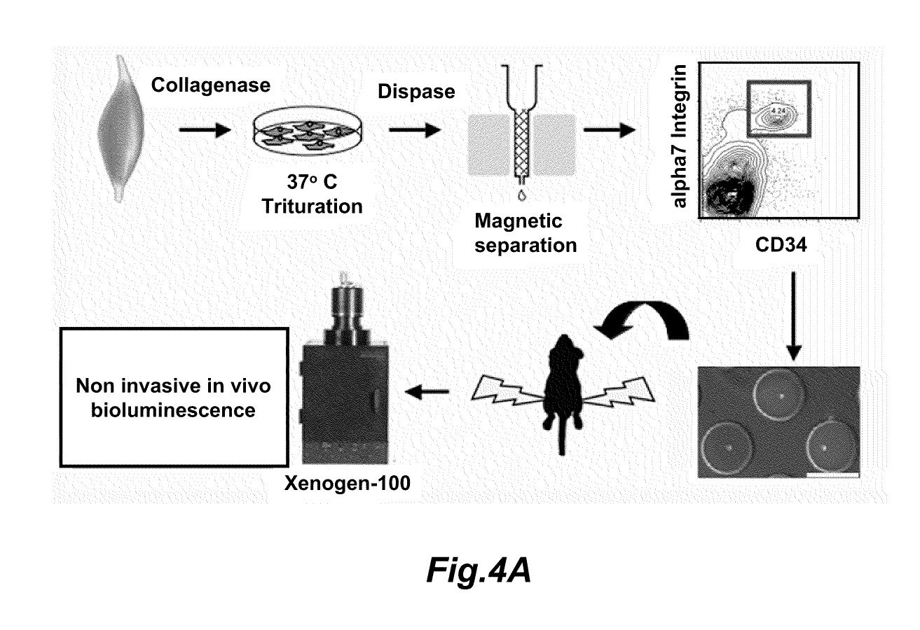

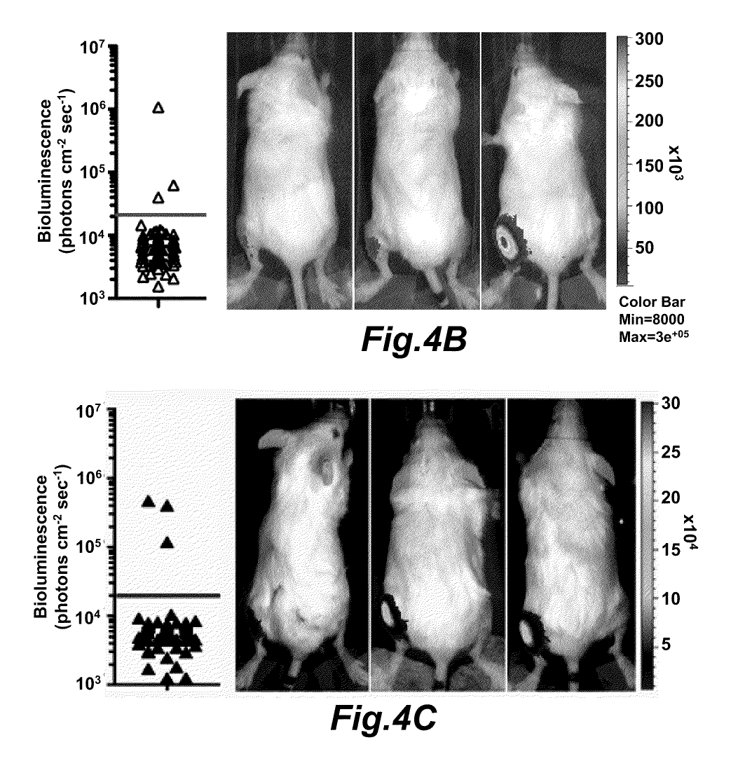

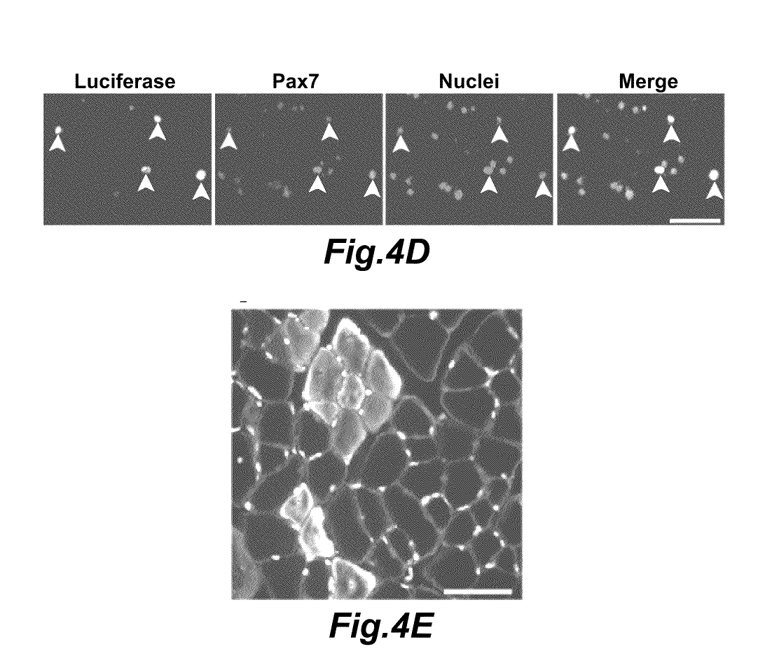

To establish that satellite cells are capable of self-renewal in vivo, transplantation and analysis of the progeny of a single cell was required. Since in the above experiments more than one cell (10-500 cells/muscle) was transplanted, it was possible that different satellite cells populations gave rise to the three successive waves of progenitor proliferation, without ever giving rise to another satellite cell. To test this possibility, satellite cells were FACS-purified and spatially segregated as single cells in very small volumes (<10 μl) in hydrogel microwells of 150 μm diameter. After 2 hours, single cells were individually picked by micromanipulation and each was injected into the irradiated TA muscle of a mouse (FIG. 4A). This resulted in 3 mice of a total of 72 transplanted with single cells (4%) that exhibited engraftment above background 4 weeks after transplantation (FIGS. 4B and 4C).

This bioluminescence assay of luciferase activity revealed that the progeny of a single adult satellite cell are capable of a high degree of proliferation during engraftment, since in the three mice each receiving a single satellite cell, a signal equivalent to 21,000, 23,000 and 84,000 cells (equivalent to about 14-17 doublings), was detected. To determine if self-renewal had occurred and not just expansion of progenitors derived from the satellite cell, the muscles were dissected. At least 50 donor-derived luciferase+cells per mouse expressing the satellite cell transcription factor, Pax7, were identified two months after transplantation of a single cell (FIG. 4D). The proliferation of single implanted muscle stem cells (muscle stem cells) in the muscle tissue of the recipient mice is shown in FIG. 4E. These results demonstrated that a single muscle satellite cell can self-renew, giving rise to a population of mononucleated Pax7⁺ cells which stably reside in recipient muscles.

The methods of the disclosure provided evidence that the muscle satellite cell is a stem cell. This required a demonstration that after transplantation, a single cell is capable of both self-renewal and the generation of more specialized progenitors. The bioluminescence imaging methods of the disclosure revealed the time-course and magnitude of the proliferative response of which satellite cells and their progeny are capable in vivo. In contrast to myoblasts, transplantation of as few as 10 satellite cells into irradiated muscles led to about 31,000 cells, followed by a plateau reflecting engraftment; a few weeks later, engrafted cells are then capable of mounting two sequential approximately 100-fold cell expansions in response to NTX injury. The plateau, or stabilization, of the signal after the transplantation into irradiated muscles is significant, and likely reflects a proliferation of cells until the need is met, following which a combination of cell death and quiescence lead to tissue homeostasis.

The overall number of satellite cell derivatives that contribute, measured as bioluminescence, may reflect both the participation of transplanted stem cells and the increasing participation of radiation resistant endogenous stem cells to tissue homeostasis over time. The plateau is reached sooner, and is higher, when larger numbers of cells are injected (FIG. 2F), whereas when fewer cells are injected, it is delayed and the magnitude is substantially lower. These results with satellite cells are in agreement with the bioluminescence response obtained with different numbers of transplanted hematopoietic stem cells (Cao et al., Proc. Natl. Acad. Sci. U.S.A. 101, 221 (2004)), and suggest that satellite stem cells, unlike transformed cells, are subject to feedback control and cease to expand when further proliferation is not necessary or desirable. The non-invasive methods of the present disclosure provide a quantitative means of assessing satellite cell behavior that will have further application in comparative studies of the dynamic regenerative potential of diverse muscle stem cell populations, assessing stem cell responses in genetic and induced models of muscle damage (exercise, freeze-injury, toxins, chemicals), and in response to therapeutic agents.

One aspect of the present disclosure, therefore, encompasses non-invasive methods for determining the proliferative status of engrafted stem cells in a recipient subject mammal, comprising: providing an isolated stem cell or a population of stem cells, wherein the stem cell or population of stem cells expresses a heterologous reporter; delivering the isolated stem cell or population of stem cells to a subject mammal; and non-invasively detecting the reporter in the recipient subject mammal, thereby detecting the population of engrafted stem cells, or progeny thereof, in the subject mammal.

In embodiments of this aspect of the disclosure, the isolated stem cell or population of stem cells may be obtained from a transgenic animal, where the transgenic animal comprises a heterologous nucleic acid encoding the reporter operably linked to a promoter driving expression of the heterologous nucleic acid.

In embodiments of this aspect of the disclosure, the step of providing an isolated stem cell or a population of stem cells can further comprise the step of transfecting a stem cell or population of stem cells with a heterologous nucleic acid encoding the reporter, wherein the reporter is operably linked to a promoter driving expression of the heterologous nucleic acid, and wherein the isolated stem cell or population of stem cells is transfected with the heterologous nucleic acid after isolation from a mammal.

In embodiments of this aspect of the disclosure, the isolated stem cell, or population of stem cells can be selected from the group consisting of: a mesenchymal stem cell, a hematopoietic stem cell, a neural crest stem cell, a placental stem cell, an embryonic stem cell, and a mesodermal stem cell. In some embodiments, the isolated stem cell, or population of stem cells, is a subset of muscle satellite cell(s) isolated from a muscle tissue.

In embodiments of this aspect of the disclosure, the reporter encoded by the heterologous nucleic acid can be a bioluminescent reporter, a fluorescent reporter, a PET reporter, or a combination thereof. In some embodiments of the disclosure, the bioluminescent reporter is a luciferase.

In other embodiments of this aspect of the disclosure, the isolated stem cell can be a single stem cell isolated from a population of cells by delivery into a microwell imprinted in a hydrogel.

In embodiments of this aspect of the disclosure, the reporter is a luciferase, and the method further comprises: administering to the subject mammal a bioluminescence initiator, whereupon interaction of the bioluminescence initiator with the luciferase causes the luciferase to emit bioluminescence; and detecting the emitted bioluminescence, thereby detecting the presence of a population of stem cells in the subject.

In this aspect of the disclosure, the isolated population of stem cells can be delivered to a solid tissue of the recipient subject mammal, or to a liquid tissue.

In these embodiments of this aspect of the disclosure, the solid tissue can be selected from the group consisting of: skeletal muscle, cardiac muscle, smooth muscle, endodermal tissue, pancreatic tissue, skin, neural tissue, and a combination thereof.

In embodiments of the methods of this aspect of the disclosure, the method may further comprise measuring the intensity of the bioluminescence, where the intensity of the bioluminescence indicates the number of stem cells in the subject mammal. In these embodiments, the method can further comprise: (i) measuring a first bioluminescence intensity; (ii) delivering to the subject mammal a test compound; and (iii) measuring a second bioluminescence intensity, where a difference in the first and the second bioluminescence intensities can indicate that the test compound modulates the proliferation of the stem cell or stem cell population delivered to the subject mammal.

In these embodiments the test compound may increase the proliferation of the stem cell or population of stem cells or decrease the proliferation of the stem cell or population of stem cells.

In embodiments of this aspect of the disclosure, the isolated stem cell or population of stem cells can be selected from the group consisting of: a single stem cell type and a plurality of stem cell types. In these embodiments, each of the stem cell types of the plurality of stem cell types may be isolated from a different donor tissue.

In some embodiments of this aspect of the disclosure, each of the stem cell types of the plurality of stem cell types may comprise a heterologous nucleic acid encoding a reporter polypeptide operably linked to a promoter driving expression of the heterologous nucleic acid, and wherein each stem cell type independently expresses a different reporter polypeptide.

Another aspect of the disclosure encompasses methods for determining the suitability of an isolated stem cell for tissue replacement, comprising: obtaining a population of isolated candidate stem cells; genetically modifying a proportion of the population of candidate stem cells with a heterologous nucleic acid encoding a reporter polypeptide, where the heterologous nucleic acid can be under the expression control of a promoter selected from the group consisting of: a constitutive promoter, an inducible promoter, a stem cell-specific promoter, and a tissue specific promoter, and wherein the heterologous nucleic acid is integrated into the genome of the cells; engrafting the genetically modified candidate stem cells to a subject mammal tissue; inducing the emission of a detectable signal by the engrafted cells in the subject mammal; and determining from the intensity of the detectable signal the degree of proliferation of said cells in the subject mammal tissue, thereby indicating the suitability of the isolated cells for tissue replacement.

Yet another aspect of the disclosure encompasses methods method for repairing muscle injury, comprising: obtaining a population of muscle satellite cells; isolating from the population of muscle satellite cells a subset population having stem cell activity and regenerative capacity by: genetically modifying a proportion of the muscle satellite cells with a heterologous nucleic acid encoding a reporter polypeptide, where the heterologous nucleic acid is under the expression control of a promoter selected from the group consisting of: a constitutive promoter, an inducible promoter, a stem cell-specific promoter, and a tissue specific promoter, and where the heterologous nucleic acid is integrated into the genome of the cells; engrafting the genetically modified muscle satellite cells to a subject mammal tissue; inducing the emission of a detectable signal by the engrafted cells in the subject mammal; determining from the intensity of the detectable signal, the degree of proliferation of said cells in the subject mammal tissue, thereby indicating the suitability of the isolated muscle satellite cells for tissue replacement; and selecting the subset of isolated muscle satellite cells having regenerative capacity and delivering said cells to a site of muscle injury in a subject mammal, whereby the subset population proliferates and differentiates into myoblasts and muscle fibers to an amount that repairs the site of the injury.

Still yet another aspect of the present disclosure encompasses methods for isolating muscle stem cells from a tissue sample, comprising: obtaining from a subject animal or human a muscle tissue sample; obtaining a population of cells in suspension from the tissue sample; contacting the population of cells in suspension with a first panel of antibody species, where each species of the first panel of antibody species selectively binds to a cell surface antigen not located on a muscle stem cell surface; partitioning the muscle cells binding to the first panel of antibodies from the population of cells in suspension; contacting the population of muscle cells in suspension with a second panel of antibody species, where each species of the second panel of antibody species selectively binds to a muscle stem cell-specific surface antigen; isolating muscle stem cells from the population of cells in suspension by partitioning cells binding to the second panel of antibodies, where the partitioned cells are muscle stem cells.

In embodiments of this aspect of the disclosure, the first panel of antibody species can comprise at least one antibody species selected from the group consisting of: an anti-CD45 antibody, an anti-CD11b antibody, an anti-CD31 antibody, and an anti-Sca1 antibody.

In embodiments of this aspect of the disclosure, the second panel of antibodies comprises an anti-α7 integrin antibody, an anti-CD34 antibody, or an anti-α7 integrin antibody and an anti-CD34 antibody.

In embodiments of this aspect of the disclosure, the antibodies of the first panel of antibodies can be each conjugated to a biotin molecule, and wherein the cells binding to the first panel of antibodies are partitioned from the cell suspension by magnetic depletion of biotin-positive cells.

In embodiments of this aspect of the disclosure, the antibodies of the second panel of antibodies can be each independently bound to a fluorescent label, where cells binding to the second panel of antibodies can be partitioned by FACS flow cytometry.

In embodiments of this aspect of the disclosure, the isolated muscle stem cells are characterized as CD45⁻, CD11b⁻, CD31⁻, Sca1⁻, α7 integrin⁺, and CD34⁺.

In embodiments of this aspect of the disclosure, the tissue sample can obtained from a transgenic animal, where the cells of the transgenic animal comprise a heterologous nucleic acid encoding a reporter polypeptide operably linked to a promoter driving expression of the heterologous nucleic acid.

In embodiments of this aspect of the disclosure, the method may further comprise isolating a single muscle stem cell from a population of isolated cells by delivery into a microwell imprinted in a hydrogel.

Yet another aspect of the disclosure encompasses an isolated muscle stem cell, or a population of isolated muscle stem cells, where the isolated muscle stem cell, or population of muscle stem cells are characterized as CD45⁻, CD11b⁻, CD31⁻, Sca1⁻, α7 integrin⁺, and CD34⁺, and where the isolated muscle stem cell, or population of muscle stem cells when implanted into a recipient animal proliferate therein to form a population of engrafted stem cells.

In embodiments of this aspect of the disclosure, the isolated muscle stem cell, or a population of isolated muscle stem cells, when implanted into a recipient subject mammal, the cells or population of cells differentiate into muscle cells.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and the present disclosure and protected by the following claims.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Examples

### Example 1

### Satellite Cell Isolation

Referring now to the schematic shown in FIG. 4A, tibialis anterior muscles of mice were subjected to enzymatic dissociation (collagenase, 0.2% and dispase 0.4 U/ml, SIGMA) for 90 min, after which non-muscle tissue was gently removed under a dissection microscope. The cell suspension was filtered through a 70 μm Nylon filter (Falcon) and incubated with the following biotinylated antibodies: CD45, CD11b, CD31 and Sca1 (BD Bioscience). Streptavidin-beads (Miltenyi Biotech) were then added to the cells together with the following antibodies: α7 integrin-Phycoerythrin (PE) and CD34-Alexa647 (BD Bioscience), after which magnetic depletion of biotin-positive cells was performed. The negative population was then fractionated twice by flow-cytometry (DIVA-Van, Becton-Dickinson). Primary myoblasts were isolated as described by Rando & Blau, J Cell Biol 125, 1275 (1994) and incorporated herein by reference in its entirety.

### Example 2

### Bioluminescence Imaging

As shown in FIG. 4A, a Xenogen-100 device was used for imaging, as described by Wehrman et al., Nat Methods 3, 295 (2006) and incorporated herein by reference in its entirety. In brief, the system comprises a light-tight imaging chamber, a charge-coupled device (CCD) camera with a cryogenic refrigeration unit and the appropriate computer system (Living-image Software; Xenogen). After intraperitoneal injection of luciferin in 100 μl of PBS (0.1 mmol/Kg body weight, Xenogen), images were acquired continuously for 30 min and then stored for subsequent analysis. Images were analyzed at 15 min after luciferin injection.

### Example 3

### Immunofluorescence and Histology

Muscle tissues were prepared for histology as described by Sacco et al., J Cell Biol 171, 483 (2005) and incorporated herein by reference in its entirety. For immunofluorescence, rabbit anti-β-galactosidase (Molecular Probes), rabbit anti-GFP (Molecular Probes), rabbit anti-luciferase (Abcam), rat anti-laminin (Upstate Technologies), mouse anti-Pax7 (Developmental Mouse Hybridoma Bank), rat anti-Ki67 (Dako), mouse anti-embryonic myosin heavy chain (F1.652)(as described in Silberstein et al., Cell 46: 1075 (1986), incorporated herein by reference in its entirety), mouse anti-myogenin (Pharmingen) and TUNEL (ApopTAG, Red kit, Chemicon) were used.

### Image Acquisition of Immunofluorescence and Histology.

Images of muscle transverse sections were acquired using an epifluorescent microscope (Axioplan2; Carl Zeiss Microimaging, Inc.), Fluor 20×/0.75 objective lens, and a digital camera (ORCA-ER C4742-95; Hamamatsu Photonics). The software used for acquisition was OpenLab 4.0.2 (Improvision). Images of cell cultures were acquired using a laser-scanning confocal microscope (LSM510; Carl Zeiss Microimaging, Inc.) using a Plan NeoFluar 20×/0.50 objective lens and maximum optical section with the LSM software. All images were composed and edited in Photoshop 7.0 (Adobe). Background was reduced using brightness and contrast adjustments, and color balance was performed to enhance colors. All the modifications were applied to the whole image using Photoshop 7.0 (Adobe).

### Example 4

### Animals.

Myf5-nLacZ transgenic mice and L2G85 (Flue) strain ubiquitously expressing luciferase from the ACTB promoter were used to generate the double transgenic animals. The NOD/SCID immunodeficient mice were purchased from the Jackson Laboratories.

### Example 5

### Cell Transplantation, Notexin Damage and Imaging.

NOD/SCID mice were anesthetized with xylazine/ketamine and shielded in a lead-jig so that only the legs were exposed to the radiation source. A single dose of 18Gy was administered to the legs and cell transplantation was performed on the same day. Freshly isolated satellite cells or primary myoblasts from the same double transgenic mice were resuspended in 2.5% goat serum in PBS and a 10 μl of cell suspension (with different cell concentrations, as indicated in the text) was injected intramuscularly into the Tibialis Anterior (TA) muscles of recipient mice. For local tissue injury, mice were anesthetized with isofluorane and a single 10 μl injection of notexin (10 g/ml, Latoxan, France) was delivered to the TAs of recipient mice.

### Example 6

### Single Cell Transplantation.

Muscle stem cells were isolated as described in Example 1 above. After isolation, to spatially segregate them, cells were plated in a well of a 24-well plate containing an array of hydrogel microwells of 150 μm diameter each (about 500 microwells/array), fabricated as described by Lutolf & Hubbel, Nature Biotech. 23: 47 (2005), incorporated herein by reference in its entirety, and placed in a 37° C. incubator. After 2 hrs, when the cells had settled at the bottom of microwells, individual cells were picked using a Narishige micromanipulator and placed in an eppendorf tube containing 10 μl of FACS buffer. Cells were then individually injected into irradiated TA muscles of NOD/SCID mouse recipients.

### Example 7

### Fabrication of Hydrogel Microwell Arrays to Probe Single HSC Biology in High-Throughput

(a) Poly(ethylene glycol) (PEG): 8arm-PEG-OH (mol. wt. 40000 g/mol) and linear PEG-(SH)₂ (mol. wt. 3400 g/mol, 100% substitution). Divinyl sulfone was purchased from Aldrich (Buchs, Switzerland). 8arm-PEG-vinylsulfones (8arm-PEG-VS) were produced and characterized as described by Lutolf & Hubbell, Biomacromolecules 4, 713 (2003), incorporated herein by reference in its entirety. The final product was dried under vacuum and stored under argon at −20° C.

The degree of end group conversion, confirmed with 1 H NMR (CDCl3): 3.6 ppm (PEG backbone), 6.1 ppm (d, 1H, ═CH2), 6.4 ppm (d, 1H, ═CH2), and 6.8 ppm (dd, 1H, −SO₂CH═), was found to be 87%.

(b) Gelation of PEG precursors: A mild and versatile chemistry described by Lutolf et al., Advanced Materials 15, 888 (2003), incorporated herein by reference in its entirety, was adapted to form hydrogels from the above PEG precursors in stoichiometrically balanced amounts. Both precursors were dissolved at a solid concentration of 10% (w/v) in 0.3 M triethanolamine (8-arm-PEG-VS) and in ultra pure water (PEG-(SH)₂), respectively, and mixed to form cross-linked gel networks by Michael-type addition.

To avoid batch-to-batch variability, each precursor solution was prepared in large quantities (of about 2.5 ml), filter sterilized (0.22 μm) and aliquoted in amounts for the synthesis of approximately 250 μl PEG hydrogel.

(c) Hydrogel microwell array formation: Hydrogel microwell arrays were fabricated by a multistep soft lithography process. PDMS microwell array replication masters of the size of an entire Si wafer were obtained. Prior to PEG gel casting, the PDMS master was cut to a size matching a desired well-format (96-, 48- or 24-well), thoroughly cleaned, and then modified with a surface layer of ₁H,₁H,₂H,₂H-perfluorodecyltrichlorosilane (Oakwood Chemicals, USA). Immediately after mixing of the above precursors in an Eppendorf tube, the PEG precursor solutions (approximately 80 μl for the 24-well size) was pipetted on the PDMS surface positioned on a hydrophobic glass slide (pre-coated with SIGMACOTE™, Sigma, USA).

Appropriate spacers of the thickness of the PDMS master plus 0.7 mm were placed at both ends of the glass slide and a second hydrophobic slide was placed on top. The two slides were fixed with binder clips on both ends, ensuring an optimal wetting of the PDMS microstructures with the precursor solution. Curing of the gel network was conducted for 30 min at 37° C. in a humidified incubator. The produced PEG hydrogel microwell arrays were peeled off using a pair of blunt forceps, washed at least 4×15 min with 4 ml PBS, and swollen overnight in PBS. Prior to cell culture, the swollen PEG hydrogel microwell arrays were fixed on the bottom of plastic wells of a desired well plate using the above gel precursor solution as efficient ‘glue’, and the arrays were equilibrated at 37° C. in cell culture medium.

### Example 8

### Cell Culture.

Cells were isolated from muscle tissue by enzymatic dissociation as described above. Cells were plated on dishes coated with Laminin (Roche) in F10/DMEM (50/50)+15% FBS+2.5 ng/ml bFGF (GM) for proliferation and in DMEM+2% horse serum (DM) for differentiation.

### Example 9

### Luciferase Activity Assay in Protein Extracts.

Myoblasts or TA myofibers were isolated from transgenic mice constitutively expressing luciferase and plated in a 24-well plate. Immediately afterwards, cells were lysed. After complete lysis, luciferin substrate (1 mM) was added to the protein extracts and bioluminescence was measured. In aliquots of the same samples, DNA was extracted, quantified with NanoDrop ND-1000 (Thermo Fisher Scientific) and luciferase activity was normalized per microgram of DNA.

### Example 10

### Cell Proliferation During Muscle Regeneration.

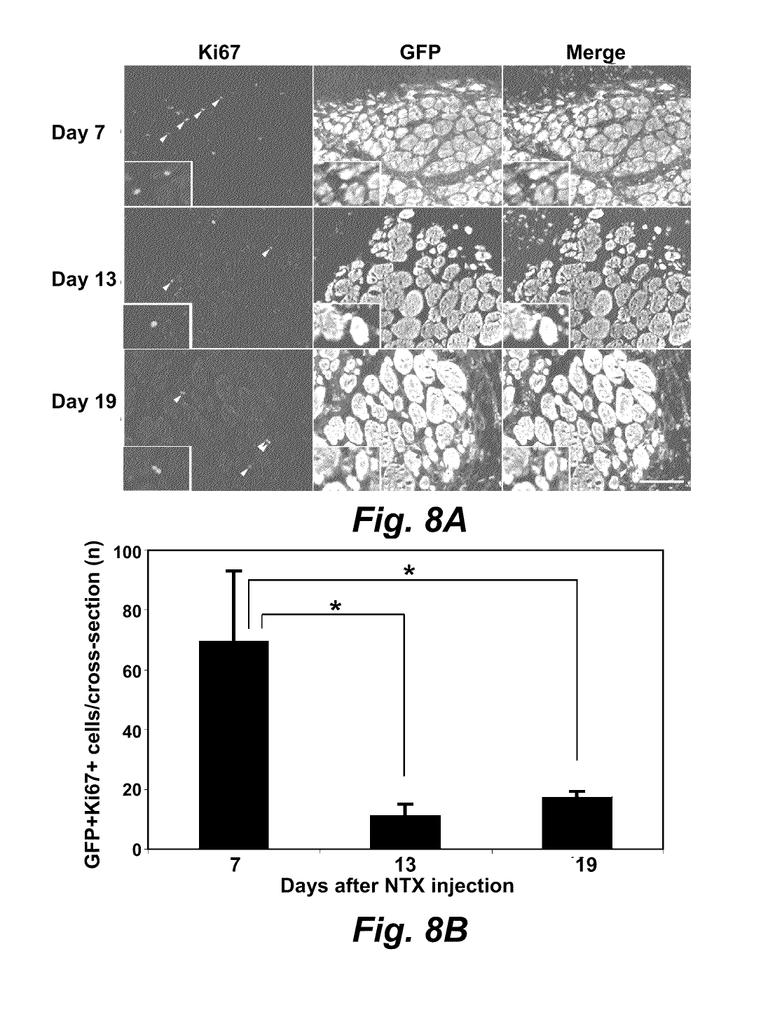

As shown in FIG. 8A, legs of NOD/SCID mice were irradiated with 18Gy and transplanted with muscle stem cells from GFP/FLuc double transgenic mice. 4 weeks later, tibialis anterior (TA) muscles were damaged with NTX and tissue harvested at the indicated days and immunostained for the proliferation marker Ki67, and for GFP. Regeneration was continuing 19 days post-damage in these experimental conditions, as shown by the presence of donor-derived (GFP⁺) proliferating cells (Ki67⁺) and small newly forming myofibers. Scale bars=80 μm.

Shown in FIG. 8B is the quantification of donor-derived (GFP⁺) proliferating (Ki67⁺) cells at day 7, 13 and 19 after NTX damage (average±s.e.m) (n=3, *P<0.05). These results show that donor-derived cells continued to proliferate and accumulate in recipient muscles for a period of at least 2-3 weeks post injury.

### Example 11

### Apoptosis Increases Over Time During NTX-Induced Muscle Regeneration

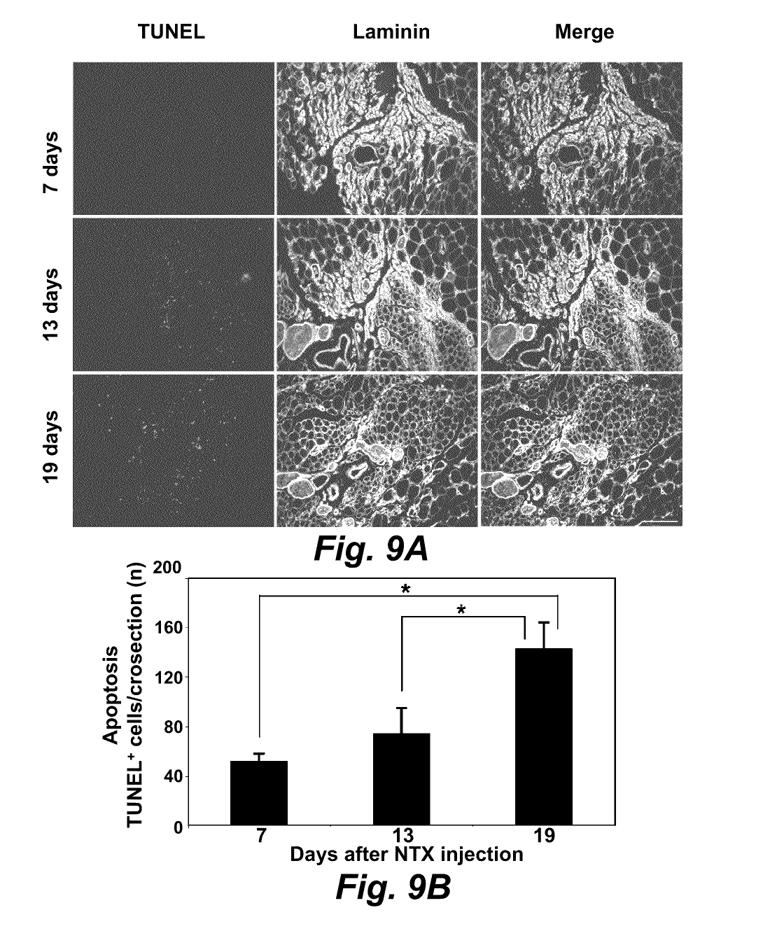

As shown in FIG. 9A, legs of NOD/SCID mice were irradiated with 18Gy and transplanted with muscle stem cells. 4 weeks later, tibialis anterior muscles were damaged with NTX and tissue harvested at the indicated days and immunostained for apoptotic cells (TUNEL) and for the basal lamina (Laminin). Apoptotic cells were visible, and they increased in number over time from 7 days to 19 days post injury, indicating a role for cell death in tissue homeostasis during regeneration. Scale bars=130 μm. FIG. 9B shows a graph showing quantification of apoptotic cells (TUNEL+) during muscle regeneration. Cell death (TUNEL+cells) progressively increased over time and was highest at day 19, when luciferase activity started decreasing (average±s.e.m) (n=4, *P<0.05).

### Example 12

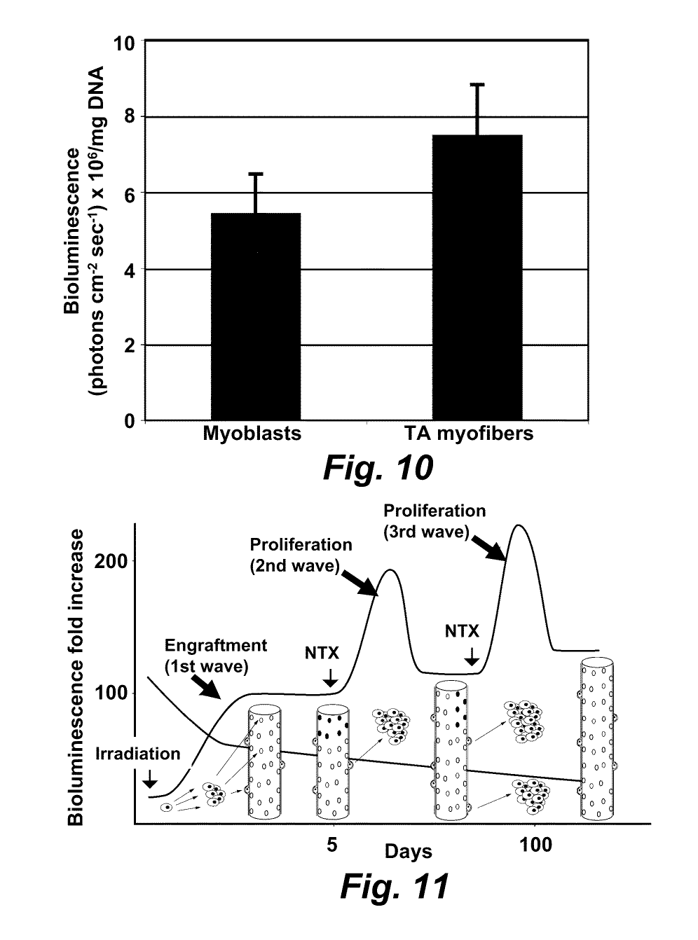

Proliferating myoblasts or tibialis anterior myofibers derived from transgenic mice expressing constitutive luciferase were lysated and assayed in a 24-well plate for luciferase activity. Aliquots of lysates were assayed for DNA content and results expressed as luciferase activity/microgram DNA. As shown in FIG. 10, luciferase activity was not significantly different between myoblasts and myofibers, indicating that this assay is a useful readout for quantifying numbers of donor-derived nuclei in transplanted muscles.

### Example 13

Schematically represented in FIG. 11 are the dynamics of muscle stem cell behavior in vivo during three waves of proliferation. Bioluminescence imaging of transplanted muscle stem cells (muscle stem cells) was indicative of their number and revealed the magnitude and kinetics of their proliferative response (FIG. 11) relative to more committed myoblasts in the same mice imaged repeatedly over time.

(a) Wave 1: Following transplant into muscles depleted of endogenous stem cells by irradiation, a first wave of approximately 100-fold expansion of cells occurs within two weeks, after which a plateau was reached, indicating that homeostasis had been achieved;

(b) Wave 2: Following injury by NTX injection, muscle stem cells underwent a second wave of rapid 80-100-fold proliferation within 2 weeks; and

(c) Wave 3: Following a second NTX injection a third wave of expansion of similar magnitude and time course was observed. Only stem cells exhibited this behavior. Myoblasts, the more specialized mononucleated progeny of stem cells, are incapable of yielding such waves of proliferation.

### Example 14

### Single Cell RT-PCR.

(a) Single cell collection: Single cells were directly sorted via FACS (Diva, BD) into PCR tubes containing 9-μl aliquots of RT-PCR lysis buffer. The buffer components included commercial RT-PCR buffer (SuperScript One-Step RTPCR Kit Reaction Buffer, Invitrogen), RNase inhibitor (Protector RNase Inhibitor, Roche) and 0.15% IGEPAL detergent (Sigma). After a short pulse-spin, the PCR tubes were immediately shock-frozen and stored at −80° C. for subsequent analysis.

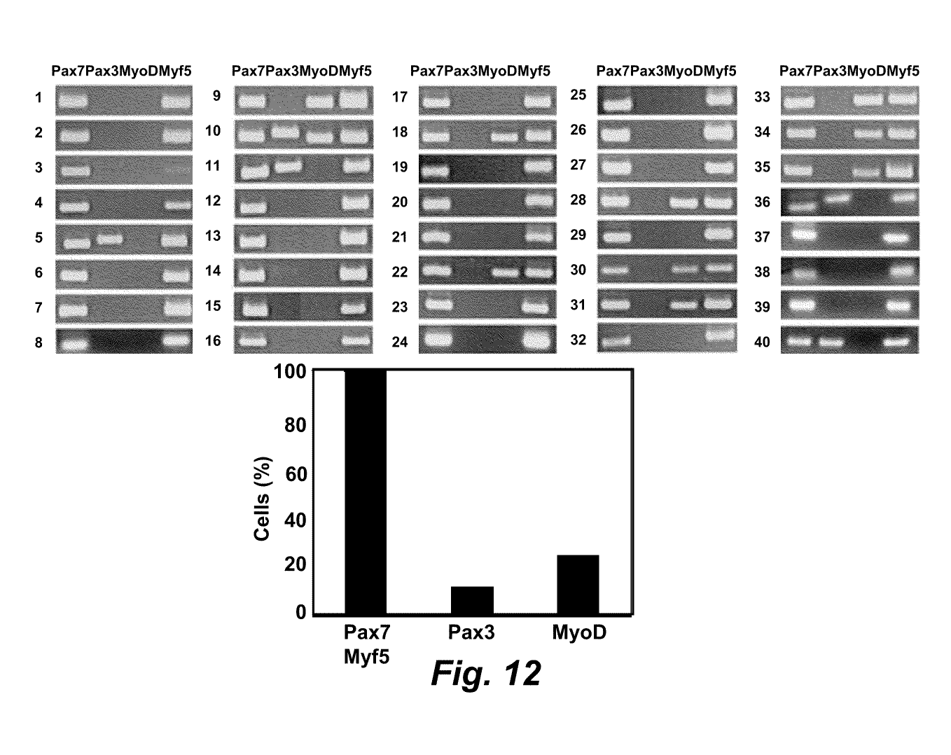

(b) Two-Step multiplex nested single cell RT-PCR: Cell lysates were first reverse transcribed using the pairs of gene-specific primers as described by the manufacturer (SuperScript One-Step RT-PCR Kit, Invitrogen). Briefly, the RT-PCR was performed in the same PCR cell-lysis tubes by addition of a RT-PCR reaction mix containing the gene-specific primer pairs and RNase inhibitor. Genomic products were excluded by designing and using intron-spanning primer sets for the first and second round PCR (see FIG. 12 and Table 1 below). Nested RT-PCR ensured greater specificity. The expected PCR-product sizes for the first and second round were approximately 450 bp (external primers) and 250 bp (internal primers), respectively.

In the first step, the reverse transcription reactions were carried out at 55° C. for 30 min, and followed by a 2-min step at 94° C. Subsequently, 30 cycles of PCR amplification were performed as follows: 94° C. for 20 sec; 60° C. for 25 sec; 68° C. for 30 sec. In the final PCR step, the reactions were incubated for 3 min at 68° C. The completed reactions were stored at 4° C. In a second step of the nested RT-PCR protocol, the completed RT-PCR reaction from the first step was diluted 1:1 with water. One percent of these reactions were replica transferred into new reaction tubes for the second round of PCR, which was performed for each of the genes separately using fully nested gene-specific internal-primers, for greater specificity, as indicated by the manufacturer in a total reaction volume of 20 μl (Platinum Taq Super-Mix HF, Invitrogen). Thirty cycles of PCR amplification were performed as follows: 94° C. for 20 sec; 60° C. for 20 sec; 68° C. for 20 sec. In the final PCR step, the reactions were incubated for 3 min at 68° C. The completed reactions were stored at 4° C. Finally, the second-round PCR products were subjected to gel electrophoresis using one fifth of the reaction volumes and 1.4% agarose gels.

TABLE 1
Primer sequences utilized for single cell PCR

| Multi- | Nested Primer Sets | |
|---|---|---|
| plex | External Primers | Internal Primers |
| genes | [5′-3′] | [5′-3′] |
| Pax7 5′ | (SEQ ID NO.: 1) | (SEQ ID NO.: 2) |
| | gaaccacatccgtcacaaga | tttcccatggttgtgtctcc |
| Pax7 3′ | (SEQ ID NO.: 3) | (SEQ ID NO.: 4) |
| | gagcactcggctaatcgaac | gtcgcagtgaccgtcctt |
| Pax3 5′ | (SEQ ID NO.: 5) | (SEQ ID NO.: 6) |
| | aaccatatccgccacaagat | aaacccaagcaggtgacaac |
| Pax3 3′ | (SEQ ID NO.: 7) | (SEQ ID NO.: 8) |
| | ctagatccgcctcctcctct | ggatgcggctgatagaactc |
| Myf5 5′ | (SEQ ID NO.: 9) | (SEQ ID NO.: 10) |
| | agacgcctgaagaaggtcaa | ccaccaaccctaaccagaga |
| Myf5 3′ | (SEQ ID NO.: 11) | (SEQ ID NO.: 12) |
| | agctggacacggagctttta | ctgttctttcgggaccagac |
| MyoD 5′ | (SEQ ID NO.: 13) | (SEQ ID NO.: 14) |
| | agcgcaagaccaccaacgct | gccttctacgcacctggac |
| MyoD 3′ | (SEQ ID NO.: 15) | (SEQ ID NO.: 16) |
| | gtggagatgcgctccactat | actcttccctggcctggact |

1. A non-invasive method for determining the proliferative status of engrafted stem cells in a recipient subject mammal, comprising:
(a) providing an isolated stem cell or a population of stem cells, wherein the stem cell or population of stem cells expresses a heterologous reporter;
(b) delivering the isolated stem cell or population of stem cells to a subject mammal; and
(c) non-invasively detecting the reporter in the recipient subject mammal, thereby detecting the population of engrafted stem cells, or progeny thereof, in the subject mammal.

2. The method of claim 1, wherein the isolated stem cell or population of stem cells is obtained from a transgenic animal, and wherein the transgenic animal comprises a heterologous nucleic acid encoding the reporter, wherein the heterologous nucleic acid is operably linked to a promoter driving expression of the heterologous nucleic acid.

3. The method of claim 1, wherein the step of providing an isolated stem cell or a population of stem cells further comprises transfecting a stem cell or population of stem cells with a vector comprising a heterologous nucleic acid encoding the reporter, wherein the reporter is operably linked to a promoter driving expression of the heterologous nucleic acid, and wherein the isolated stem cell or population of stem cells is transfected with the heterologous nucleic acid after isolation from a subject mammal.

4. The method of claim 1, wherein the isolated stem cell, or population of stem cells is selected from the group consisting of: a mesenchymal stem cell, a hematopoietic stem cell, a neural crest stem cell, a placental stem cell, an embryonic stem cell, and a mesodermal stem cell.

5. The method of claim 1, wherein the isolated stem cell, or population of stem cells, is a subset of muscle satellite cell(s) isolated from a muscle tissue.

6. The method of claim 1, wherein the reporter encoded by the heterologous nucleic acid is selected from the group consisting of: a bioluminescent reporter, a fluorescent reporter, a PET reporter, and a combination thereof.

7. The method of claim 1, wherein the bioluminescent reporter is a luciferase.

8. The method of claim 1, wherein the isolated stem cell is a single stem cell isolated from a population of cells by delivery into a microwell imprinted in a hydrogel.

9. The method of claim 1, wherein the reporter is a luciferase, and the method further comprises:
administering to the subject mammal a bioluminescence initiator, whereupon interaction of the bioluminescence initiator with the luciferase causes the luciferase to emit bioluminescence; and
detecting the emitted bioluminescence, thereby detecting the presence of a population of stem cells in the subject.

10. The method of claim 1, wherein the isolated population of stem cells is delivered to a solid tissue of the recipient subject mammal, or to a liquid tissue.

11. The method of claim 10, wherein the solid tissue is selected from the group consisting of: skeletal muscle, cardiac muscle, smooth muscle, endodermal tissue, pancreatic tissue, skin, neural tissue, and a combination thereof.

12. The method of claim 1, further comprising the step of measuring the intensity of the bioluminescence, wherein the intensity of the bioluminescence indicates the number of stem cells in the subject mammal.

13. The method of claim 12, further comprising:
(i) measuring a first bioluminescence intensity;
(ii) delivering to the subject mammal a test compound; and
(iii) measuring a second bioluminescence intensity, whereby a difference in the first and the second bioluminescence intensities indicates that the test compound modulates the proliferation of the stem cell or stem cell population delivered to the subject mammal.

14. The method of claim 13, wherein the test compound increases the proliferation of the stem cell or population of stem cells.

15. The method of claim 13, wherein the test compound decreases the proliferation of the stem cell or population of stem cells.

16. The method of claim 1, wherein the isolated population of stem cells comprises a plurality of stem cell types.

17. The method of claim 16, wherein each of the stem cell types of the plurality of stem cell types is isolated from a different donor tissue.

18. The method of claim 16, wherein each of the stem cell types of the plurality of stem cell types comprises a heterologous nucleic acid encoding a reporter polypeptide operably linked to a promoter driving expression of the heterologous nucleic acid, and wherein each stem cell type independently expresses a different reporter polypeptide.

19. A method for determining the suitability of an isolated stem cell for tissue replacement, comprising:
(i) obtaining a population of isolated candidate stem cells;
(ii) genetically modifying a proportion of the population of candidate stem cells with a heterologous nucleic acid encoding a reporter polypeptide, wherein the heterologous nucleic acid is under the expression control of a promoter selected from the group consisting of: a constitutive promoter, an inducible promoter, a stem cell-specific promoter, and a tissue specific promoter, and wherein the heterologous nucleic acid is integrated into the genome of the cells;
(iii) engrafting the genetically modified candidate stem cells to a subject mammal tissue;
(iv) inducing the emission of a detectable signal by the engrafted cells in the subject mammal; and
(v) determining from the intensity of the detectable signal, the degree of proliferation of said cells in the subject mammal tissue, thereby indicating the suitability of the isolated stem cells for tissue replacement.

20. A method for repairing muscle injury, comprising:
(a) obtaining a population of muscle satellite cells;
(b) isolating from the population of muscle satellite cells a subset population having stem cell activity and regenerative capacity by:
(i) genetically modifying a proportion of the muscle satellite cells with a heterologous nucleic acid encoding a reporter polypeptide, wherein the heterologous nucleic acid is under the expression control of a promoter selected from the group consisting of: a constitutive promoter, an inducible promoter, a stem cell-specific promoter, and a tissue specific promoter, and wherein the heterologous nucleic acid is integrated into the genome of the cells;
(ii) engrafting the genetically modified muscle satellite cells to a subject mammal tissue;
(iii) inducing the emission of a detectable signal by the engrafted cells in the subject mammal; and
(iv) determining from the intensity of the detectable signal, the degree of proliferation of said cells in the subject mammal tissue, thereby indicating the suitability of the isolated muscle satellite cells for tissue replacement;
(c) selecting the subset of isolated muscle satellite cells having regenerative capacity and delivering said cells to a site of muscle injury in a subject mammal, whereby the subset population proliferates and differentiates into myoblasts and muscle fibers to an amount that repairs the site of the injury.

21. A method for isolating muscle stem cells from a tissue sample, comprising:
(a) obtaining from a subject animal or human a muscle tissue sample;
(b) obtaining a population of cells in suspension from the tissue sample;
(c) contacting the population of cells in suspension with a first panel of antibody species, wherein each species of the first panel of antibody species selectively binds to a cell surface antigen not located on a muscle stem cell surface;
(d) partitioning the muscle cells binding to the first panel of antibodies from the population of cells in suspension;
(e) contacting the population of muscle cells in suspension with a second panel of antibody species, wherein each species of the second panel of antibody species selectively binds to a muscle stem cell-specific surface antigen; and
(f) isolating muscle stem cells from the population of cells in suspension by partitioning cells binding to the second panel of antibodies, wherein the partitioned cells are muscle stem cells.

22. The method of isolating muscle stem cells of claim 21, wherein the first panel of antibody species is selected from the group consisting of: an anti-CD45 antibody, an anti-CD11b antibody, an anti-CD31 antibody, and an anti-Sca1 antibody.

23. The method of isolating muscle stem cells of claim 21, wherein the second panel of antibodies comprises an anti-α7 integrin antibody, an anti-CD34 antibody, or a combination of an anti-α7 integrin antibody and an anti-CD34 antibody.

24. The method of isolating muscle stem cells of claim 21, wherein the antibodies of the first panel of antibodies are each conjugated to a biotin molecule, and wherein the cells binding to the first panel of antibodies are partitioned from the cell suspension by magnetic depletion of biotin-positive cells.

25. The method of isolating muscle stem cells of claim 21, wherein the antibodies of the second panel of antibodies are each bound to a fluorescent label, and wherein cells binding to the second panel of antibodies are partitioned by FACS flow cytometry.

26. The method of isolating muscle stem cells of claim 21, wherein the isolated muscle stem cells are characterized as CD45⁻, CD11b⁻, CD31⁻, Sca1⁻, α7 integrin⁺, and CD34⁺.

27. The method of isolating muscle stem cells of claim 21, wherein the tissue sample is obtained from a transgenic animal, wherein the cells of the transgenic animal comprise a heterologous nucleic acid encoding a reporter polypeptide operably linked to a promoter driving expression of the heterologous nucleic acid.

28. The method of isolating muscle stem cells of claim 21, further comprising isolating a single muscle stem cell from a population of isolated cells by delivery into a microwell imprinted in a hydrogel.

29. An isolated muscle stem cell, or a population of isolated muscle stem cells, wherein the isolated muscle stem cell, or population of muscle stem cells are characterized as CD45⁻, CD11b⁻, CD31⁻, Sca1⁻, α7 integrin⁺, and CD34⁺, and wherein the isolated muscle stem cell, or population of muscle stem cells when implanted into a recipient mammal proliferate therein to form a population of engrafted stem cells.

30. The isolated muscle stem cell or population of isolated muscle stem cells of claim 29, wherein when implanted into a recipient subject mammal, the cells or population of cells differentiate into muscle cells.
